(19) United States
(12) Patent Application Publication
(10) Pub. No.: US 2018/0136208 A1
(43) Pub. Date: May 17, 2018

(54) Biochemical Mediators of Platelet Storage

(71) Applicants: James Charles Zimring, Seattle, WA (US); Sherrill J. Slichter, Seattle, WA (US); Xiaoyun Fu, Seattle, WA (US); Jake Felcyn, Seattle, WA (US); Bloodworks, Seattle, WA (US)

(72) Inventors: James Charles ZIMRING, Seattle, WA (US); Sherrill J. SLICHTER, Seattle, WA (US); Xiaoyun FU, Kenmore, WA (US); Jake FELCYN, Seattle, WA (US)

(21) Appl. No.: 15/564,414

(22) PCT Filed: Apr. 7, 2016
(86) PCT No.: PCT/US2016/026530

§ 371 Date: Oct. 4, 2017

Related U.S. Application Data
(60) Provisional application No. 62/144,084, filed on Apr. 7, 2015.

(51) Int. Cl. G01N 33/569; G01N 33/49; A61K 35/16; A61K 35/19; G16H 10/40

(52) U.S. Cl. CPC G01N 33/56966; G01N 33/49; G01N 2030/025; A61K 35/19; G16H 10/40; A61K 35/16

(57) ABSTRACT

Compositions and methods for determining post-transfusion survival of platelets and the suitability of platelet units for transfusion by measuring the levels of one or more biochemical mediators in a platelet sample are provided.

### FIELD

The invention relates to compositions and methods for determining post-transfusion survival of platelets (PLT), efficacy of PLTs. and potential untoward toxicities of PLTs, by measuring the levels of one or more biochemical mediators in a PLT sample.

### BACKGROUND

In excess of 4,000,000 units of platelets (PLTs) are transfused annually in the United States. Currently, there are only 3 quality control measures utilized prior to release of a unit of PLTs: 1) the absence of screened pathogens, 2) visual assessment for swirling and the presence of visual abnormalities suggestive of bacterial contamination (with or without formal bacterial screening), 3) storage history of agitation and temperature control. However, it has been known for decades that the quality of PLTs can vary widely from unit to unit and from donor to donor. Indeed, transfusion of certain units may result in substantial increases in recipient circulating platelets, whereas other units give no discernable benefit. However, the factors that regulate whether PLTs collected from a given donor store well or not is poorly understood. For this reason, currently, there are no quality control measures related to the extent to which a transfused unit of PLTs will survive post-transfusion. This is a medical problem since PLTs that survive poorly post-transfusion result in a less efficacious product from the standpoint of PLT replacement. Collection and transfusing of PLTs is an expensive and time consuming process, and the inability to distinguish which units and/or which donors will not result in an efficacious unit results in a substantial waste of medical resources.

Disclosed herein is a method for assessing a PLT unit (prior to transfusion) allowing the prediction of its post-transfusion survival and also potential toxicity. Specifically, biochemical mediators that predict if PLTs will survive well post-transfusion are presented herein.

### SUMMARY

Described herein are compositions and methods for determining post-transfusion survival of a platelet (PLT) unit by measuring the levels of one or more biochemical mediators in a PLT sample.

In a first aspect, disclosed herein is a method of determining post-transfusion survival of platelets (PLT) prior to transfusion, the method comprising the steps of: a) measuring the levels of one or more biochemical mediators in a PLT sample selected from the group consisting of the compounds in Tables 2-5; b) comparing the level of the one or more biochemical mediators in the PLT sample with the level of the one or more biochemical mediators present in a control sample; c) determining a higher or lower level of the one or more biochemical mediators in the PLT sample that indicates the post-transfusion survival of platelets in the PLT sample.

In some embodiments, the method further comprises selecting the PLT sample for use in a transfusion when a higher post-transfusion survival is indicated.

In some embodiments, the method further comprises excluding the PLT sample for use in a transfusion when a lower post-transfusion survival is indicated.

In a second aspect, disclosed herein is a method of determining the suitability of a platelet (PLT) unit for transfusion, the method comprising the steps of: a) measuring the levels of one or more biochemical mediators in a PLT sample selected from the group consisting of the compounds in Tables 2-5; b) comparing the level of the one or more biochemical mediators in the PLT sample with the level of the one or more biochemical mediators present in a control sample; c) determining a higher or lower level of the one or more biochemical mediators in the PLT sample that indicates the suitability of the platelet (PLT) unit for transfusion.

In some embodiments, the method further comprises selecting the PLT sample for use in a transfusion when a higher suitability of the platelet (PLT) unit for transfusion is indicated.

In some embodiments, the method further comprises excluding the PLT sample from use in a transfusion when a lower suitability of the platelet (PLT) unit for transfusion is indicated.

In an embodiment, the levels of the one or more biochemical mediators in the PLT sample is indicative of the level of leukotrienes or prostaglandins in the PLT sample, thereby indicating the suitability of the sample for transfusion.

In various embodiments of the first and second aspects, the measurement is performed at the time of collection of the PLT sample.

In various embodiments of the first and second aspects, the measurement is performed during the time of storage of the PLT sample.

In various embodiments of the first and second aspects, the measurement is performed by mass spectrometry. In various embodiments, the mass spectrometry is gas-chromatography/mass spectrometry (GC/MS) or liquid chromatography-tandem mass spectrometry (LC/MS/MS).

In various embodiments of the first and second aspects, the measurement is performed by enzymatic assay.

In various embodiments of the first and second aspects, the measurement is performed by ELISA.

In various embodiments of the first and second aspects, the level of the one or more biochemical mediator is 2-200 fold higher than in the control sample.

In a third aspect, disclosed herein is method for determining PLT storage quality, the method comprising the steps of: obtaining a dataset associated with a sample of stored platelets, wherein the dataset comprises at least one biochemical mediator, selected from the group consisting of the compounds in Tables 2-5; analyzing the dataset to determine data for the at least one biochemical mediator, wherein the data is positively correlated or negatively correlated with PLT storage quality of the sample of stored platelets.

In a fourth aspect, disclosed herein is method for determining PLT storage quality, the method comprising the steps of: obtaining a sample of stored platelets, wherein the sample comprises at least one biochemical mediator, selected from the group consisting of the compounds in Tables 2-5; contacting the sample with a reagent; generating a complex between the reagent and the at least one biochemical mediator; detecting the complex to obtain a dataset associated with the sample, wherein the dataset comprises expression or activity level data for the at least one biochemical mediator; and analyzing the expression or activity level data for the at least one biochemical mediator, wherein the expression or activity level of the at least one biochemical mediator is positively correlated or negatively correlated with PLT storage quality.

In a fifth aspect, disclosed herein is computer-implemented method for determining PLT storage quality, the method comprising the steps of: storing, in a storage memory, a dataset associated with a stored platelet sample, wherein the dataset comprises data for at least one biochemical mediator, selected from the group consisting of the compounds in Tables 2-5; and analyzing, by a computer processor, the dataset to determine the expression or activity levels of the at least one biochemical mediator, wherein the expression or activity levels are positively correlated or negatively correlated with PLT storage quality.

In a sixth aspect, disclosed herein is system for determining PLT storage quality, the system comprising: a storage memory for storing a dataset associated with a stored platelet sample, wherein the dataset comprises data for at least one biochemical mediator, wherein the dataset comprises data for at least one biochemical mediator, selected from the group consisting of the compounds in Tables 2-5; and a processor communicatively coupled to the storage memory for analyzing the dataset to determine the activity or expression levels of the at least one biochemical mediator, wherein the activity or expression levels are positively correlated or negatively correlated with PLT storage quality.

In a seventh aspect, disclosed herein is computer-readable storage medium storing computer-executable program code, the program code comprising: program code for storing a dataset associated with a stored platelet sample, wherein the dataset comprises data for at least one biochemical mediator, wherein the dataset comprises data for at least one biochemical mediator, selected from the group consisting of the compounds in Tables 2-5; and program code for analyzing the dataset to determine the activity or expression levels of the at least one biochemical mediator, wherein the activity or expression levels of the biochemical mediators are positively correlated or negatively correlated with PLT storage quality.

In an eighth aspect, disclosed herein is method for predicting transfusion outcome, the method comprising the steps of: obtaining a dataset associated with a sample of stored platelets, wherein the dataset comprises at least one biochemical mediator, wherein the dataset comprises data for at least one biochemical mediator, selected from the group consisting of the compounds in Tables 2-5; analyzing the dataset to determine data for the at least one biochemical mediator, wherein the data is positively correlated or negatively correlated with transfusion outcome if the platelet sample is transfused into a patient.

In a ninth aspect, disclosed herein is method for predicting transfusion outcome, the method comprising the steps of: obtaining a sample of stored platelets, wherein the sample comprises at least one biochemical mediator, wherein the dataset comprises data for at least one biochemical mediator, selected from the group consisting of the compounds in Tables 2-5; contacting the sample with a reagent; generating a complex between the reagent and the at least one biochemical mediator; detecting the complex to obtain a dataset associated with the sample, wherein the dataset comprises expression or activity level data for the at least one biochemical mediator; and analyzing the expression or activity level data for the biochemical mediators, wherein the expression or activity level of the at least one biochemical mediator is positively correlated or negatively correlated with transfusion outcome if the platelet sample is transfused into a patient.

In a tenth aspect, disclosed herein is computer-implemented method for predicting transfusion outcome, the method comprising the steps of: storing, in a storage memory, a dataset associated with a stored platelet sample, wherein the dataset comprises data for at least one biochemical mediator wherein the dataset comprises data for at least one biochemical mediator, selected from the group consisting of the compounds in Tables 2-5; and analyzing, by a computer processor, the dataset to determine the expression or activity levels of the at least one biochemical mediator, wherein the expression or activity levels are positively correlated or negatively correlated with transfusion outcome if the platelet sample is transfused into a patient.

In an eleventh aspect, disclosed herein is system for predicting transfusion outcome, the system comprising: a storage memory for storing a dataset associated with a stored platelet sample, wherein the dataset comprises data for at least one biochemical mediator, wherein the dataset comprises data for at least one biochemical mediator, selected from the group consisting of the compounds in Tables 2-5; and a processor communicatively coupled to the storage memory for analyzing the dataset to determine the activity or expression levels of the at least one biochemical mediator, wherein the activity or expression levels are positively correlated or negatively correlated with transfusion outcome if the platelet sample is transfused into a patient.

In a twelfth aspect, disclosed herein is computer-readable storage medium storing computer-executable program code, the program code comprising: program code for storing a dataset associated with a stored platelet sample, wherein the dataset comprises data for at least one biochemical mediator, selected from the group consisting of the compounds in Tables 2-5; and program code for analyzing the dataset to determine the activity or expression levels of the at least one biochemical mediator, wherein the activity or expression levels of the biochemical mediators are positively correlated or negatively correlated with transfusion outcome if the platelet sample is transfused into a patient.

In various embodiments of the above. The method of claim 11, wherein the sample of stored platelets is selected for use in a transfusion when the data is is positively correlated correlated with PLT storage quality of the sample of stored platelets.

In various embodiments of the above, the sample of stored platelets is selected for use in a transfusion when the data is is positively correlated correlated with PLT storage quality of the sample of stored platelets.

In various embodiments of the above, the sample of stored platelets is excluded from use in a transfusion when the data is is negatively correlated correlated with PLT storage quality of the sample of stored platelets.

In various embodiments of the above, the sample of stored platelets is selected for use in a transfusion when the data is positively correlated with transfusion outcome if the sample of stored platelets is transfused into a patient.

In various embodiments of the above, the sample of stored platelets is excluded from use in a transfusion when the data is negatively correlated with transfusion outcome if the sample of stored platelets is transfused into a patient.

In various embodiments of the above aspects, the dataset is obtained at the time of collection of the PLT sample.

In various embodiments of the above aspects, the dataset is obtained during the time of storage of the PLT sample.

In various embodiments of the above aspects, the dataset is obtained by mass spectrometry.

In various embodiments of the above aspects, the mass spectrometry is gas-chromatography/mass spectrometry (GC/MS) or liquid chromatography-tandem mass spectrometry (LC/MS/MS).

In various embodiments of the above aspects, the dataset is obtained by enzymatic assay.

In various embodiments of the above aspects, the dataset is obtained by ELISA.

In a thirteenth aspect, disclosed herein is kit for use in predicting transfusion outcome or platelet (PLT) storage quality, the kit comprising: a set of reagents comprising a plurality of reagents for determining from a stored platelet sample data for at least one biochemical mediator, selected from the group consisting of the compounds in Tables 2-5; and instructions for using the plurality of reagents to determine data from the stored platelet sample.

### BRIEF DESCRIPTION OF THE DRAWINGS

FIG. 1: Analytes and Pathways that Correlate with Platelet Recoveries and Survival. The individual analytes that correlated with PLT recoveries and survival, and their general metabolic pathways, are shown in panels (A) and (B), respectively; all values shown are for day 5 of storage. Analyte/Pathways that negatively correlate are shown in dark shading; those that correlate positively are shown in light shading. The extent of correlation, p values, and q values are shown in Tables 4 and 5, respectively. Permutation analysis of multiple members of a common pathway are presented in the main text.

FIG. 2: Negative Correlation of Caffeine Metabolites to PLT Recoveries. Quantitation of 15 caffeine metabolites is presented across the collected PLT units on day 5 (A). Correlations of caffeine and indicated caffeine metabolites using the log transformed concentrations vs. normalized recoveries (B). All calculations are Pearson correlations. Simplified metabolic pathways of Caffeine metabolism; metabolites in bold have significant negative correlations to PLT recoveries (C).

### DETAILED DESCRIPTION

The present invention generally relates to compositions and methods for determining post-transfusion survival of platelets (PLT) by measuring the levels of one or more biochemical mediators in a PLT sample.

Transfusion of platelets (PLTs) is a mainstay of therapy for patients who are thrombocytopenic¹. In addition, PLT transfusion can be an essential component of treating acute hemorrhage in trauma and/or surgical patients². Decades of research on the cellular biology and biochemistry of stored PLTs has led to a number of improvements in PLT collection and storage. Indeed, certain criteria have been established by which quality of stored PLT products can be assessed³. PLTs that have a pH drop below 6.2 have poor survival post-transfusion and likely provide little hemostatic effect; pH does not correlate with post-transfusion viability when pH is greater than 6.2⁴. Expression of surface markers (CD42b and P-selectin) as well as cellular morphology and extent of shape change (ESC) are associated with post-transfusion PLT survival⁵⁻⁷. However, even these criteria correlate relatively weakly to PLT viability post-transfusion, which is substantial from donor-to-donor⁸. The basis for donor-to-donor variation in post-transfusion viability of stored PLTs remains poorly understood.

Disclosed herein is a metabolomics approach to the issue of donor variability in post-storage PLT viability. Apheresis PLTs were collected from 21 normal subjects and PLTs were sampled longitudinally during storage. At the end of 5 day storage, post-transfusion viability was determined by autologous infusion of radiolabeled PLTs. Metabolomic analysis of the stored PLTs identified multiple specific metabolites that correlated with either PLT recoveries or survivals post-transfusion, which are detailed below. Major general pathways were also identified. Lipid metabolism components in general, and products of both lipid oxidation and acylcarnitines in particular, emerged as negative correlates of post-storage circulation and survival of transfused PLTs (PLT survival). In addition, caffeine and its metabolites emerged as potential negative correlates with PLTs circulating immediately after infusion (PLT recoveries). Together, these findings provide novel insight into the biology of PLT storage, and for the first time, correlate specific metabolites with the ability of PLTs to circulate post-transfusion.

It is to be understood that this invention is not limited to particular methods, reagents, compounds, compositions or biological systems, which can, of course, vary. It is also to be understood that the terminology used herein is for the purpose of describing particular aspects only, and is not intended to be limiting. As used in this specification and the appended claims, the singular forms “a”, “an” and “the” include plural references unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably 0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

An “analyte” or “target” refers to a compound to be detected. Such compounds can include small molecules, peptides, proteins, nucleic acids, as well as other chemical entities. In the context of the present invention, an analyte or target will generally correspond to the biochemical compounds disclosed herein, or a reaction product thereof.

The term “biomarker” refers to a molecule (typically small molecule, protein, nucleic acid, carbohydrate, or lipid) that is expressed and/or released from a cell, which is useful for identification or prediction. Such biomarkers are molecules that can be differentially expressed, e.g., overexpressed or underexpressed, or differentially released in response to varying conditions (e.g., storage). In the context of the present disclosure, this may refer to biochemical compounds, which are elevated or reduced in stored versus non-stored platelets, for instance, 1-fold, 2-fold, 3-fold, 4-fold, 5-fold or more in stored platelets versus non-stored platelets.

The term “biochemical mediator” refers to a molecule (typically a small chemical molecule, which may include, but are not limited to, peptides, nucleic acids, carbohydrates, lipids or metabolites), which are expressed and/or released from cells (including platelets), which is useful as a predictor of how units of platelets will perform upon transfusion and/or have the ability to induce biological responses through the intrinsic activities of the biochemical mediator itself. Such biochemical mediators are molecules that can be differentially expressed, e.g., overexpressed or underexpressed, or differentially released in response to varying conditions (e.g., storage). In the context of the present disclosure, this may refer to biochemical compounds, which are elevated or reduced in stored versus non-stored platelets, for instance, 1-fold, 2-fold, 3-fold, 4-fold, 5-fold or more in stored platelets versus non-stored platelets.

A “sample” refers to any source which is suspected of containing an analyte or target molecule. Examples of samples which may be tested using the present invention include, but are not limited to, blood, serum, plasma, urine, saliva, cerebrospinal fluid, lymph fluids, tissue and tissue and cell extracts, cell culture supernantants, among others. A sample can be suspended or dissolved in liquid materials such as buffers, extractants, solvents, and the like. In the context of the present application, a sample is generally a stored platelet sample of varying length of storage.

“Antibody” refers to any immunoglobulin or intact molecule as well as to fragments thereof that bind to a specific epitope that may be used in the practice of the present invention. Such antibodies include, but are not limited to polyclonal, monoclonal, chimeric, humanized, single chain, Fab, Fab′, F(ab)′ fragments and/or F(v) portions of the whole antibody and variants thereof. All isotypes are encompassed by this term and may be used in the practice of this invention, including IgA, IgD, IgE, IgG, and IgM.

An “antibody fragment” refers specifically to an incomplete or isolated portion of the full sequence of the antibody which retains the antigen binding function of the parent antibody and may also be used in the present invention. Examples of antibody fragments include Fab, Fab′, F(ab′)2, and Fv fragments; diabodies; linear antibodies; single-chain antibody molecules; and multispecific antibodies formed from antibody fragments.

An intact “antibody” for use in the invention comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2. FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind. Examples of binding include (i) a Fab fragment, a monovalent fragment consisting of the V_{L}, V_{H}, C_{L} and CH 1 domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the V_{L} and V_{H} domains of a single arm of an antibody, (v) a dAb fragment (Ward et al., Nature, 341:544-546 (1989)), which consists of a VH domain; and (vi) an isolated complementarity determining region (CDR).

“Single chain antibodies” or “single chain Fv (scFv)” may also be used in the present invention. This term refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv): see, e.g., Bird et al., Science, 242:423-426 (1988); and Huston et al., Proc Natl Acad Sci USA, 85:5879-5883 (1988)). Such single chain antibodies are included by reference to the term “antibody” fragments can be prepared by recombinant techniques or enzymatic or chemical cleavage of intact antibodies.

A “monoclonal antibody” may be used in the present invention. Monoclonal antibodies are a preparation of antibody molecules of single molecular composition. A monoclonal antibody composition displays a single binding specificity and affinity for a particular epitope.

In one embodiment, the antibody or fragment is conjugated to an “effector” moiety. The effector moiety can be any number of molecules, including labeling moieties such as radioactive labels or fluorescent labels.

A “label” or a “detectable moiety” is a composition detectable by spectroscopic, photochemical, biochemical, immunochemical, chemical, or other physical means. For example, useful labels include ³²p, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

Samples of platelets stored for various amounts of time are compared to “control” samples which can be freshly drawn platelets or platelets which have been minimally stored. Control samples are assigned a relative analyte amount or activity to which sample values are compared. Relevant levels of analyte elevation occur when the sample amount or activity value relative to the control is 100% (i.e., the same), 110%, more preferably 150%, more preferably 200-500% (i.e., two to five fold higher relative to the control), more preferably 1000-3000% higher.

As used herein, “PLT storage quality” is defined as the extent of post-transfusion recovery of the stored PLTs; higher recovery is defined as higher quality. Examples of post-transfusion recovery include greater than zero and almost 100% recovery, i.e., recovery of 10%, 20%, 30%, 40%, 50%, 60%, 70%, 80%, 90%, 100%, and all percentages in between. In general, a platelet sample has a higher PLT storage quality if it expresses elevated levels of a biochemical mediator that positively correlates with platelet survival or recovery, or lowered levels of a biochemical mediator that negatively correlates with platelet survival or recovery. Conversely, a platelet sample has a lower PLT storage quality if it expresses lowered levels of a biochemical mediator that positively correlates with platelet survival or recovery, or elevated levels of a biochemical mediator that negatively correlates with platelet survival or recovery.

As used herein. “toxicity” of a PLT unit is defined as any adverse reaction associated with transfusion of a PLT unit, including, but not limited to, fever, inflammation, induction of recipient cytokines, transfusion induced lung injury, and transfusion-related immunomodulation, among others.

As used herein, a PLT unit is less suitable for transfusion if it has lower PLT quality (i.e., post-transfusion survival) or elevated toxicity as compared to other PLT units, e.g., as compared to a control. In general, a PLT unit is more suitable for transfusion if it expresses elevated levels of a biochemical mediator that positively correlates with platelet survival or recovery, or lowered levels of a biochemical mediator that negatively correlates with platelet survival or recovery. Conversely, a PLT unit is less suitable for transfusion if it expresses lowered levels of a biochemical mediator that positively correlates with platelet survival or recovery, or elevated levels of a biochemical mediator that negatively correlates with platelet survival or recovery.

As used herein, “transfusion outcome” refers to post-transfusion survival of platelets in the circulation and the presence or absence of toxicity after platelet transfusion.

Assays for many of the biochemical compounds disclosed herein are known or commercially available.

For example, antibody reagents can be used in assays to detect the levels of analytes in platelet samples using any of a number of immunoassays known to those skilled in the art.

Immunoassay techniques and protocols are generally described in Price and Newman, “Principles and Practice of Immunoassay.” 2nd Edition, Grove's Dictionaries, 1997; and Gosling, “Immunoassays; A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (META); immunohistochemical (IHC) assays; capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to proteins can be detected directly or indirectly. Direct labels include fluorescent or luminescent tags, metals, dyes, radionuclides, and the like, attached to the antibody. A chemiluminescence assay using a chemiluminescent antibody specific for the protein is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate, a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

In some embodiments, the measurement of the biochemical mediators of the present invention is performed using various mass spectrometry methods. As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 1999, 2: 264-76; and Merchant and Weinberger, Electrophoresis 2000, 21; 1164-67.

As used herein, the term “gas chromatography” or “GC” refers to chromatography in which the sample mixture is vaporized and injected into a stream of carrier gas (as nitrogen or helium) moving through a column containing a stationary phase composed of a liquid or a particulate solid and is separated into its component compounds according to the affinity of the compounds for the stationary phase.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography).

In some embodiments, the present invention is practiced using computer implementation. In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

The following examples of specific aspects for carrying out the present invention are offered for illustrative purposes only, and are not intended to limit the scope of the present invention in any way.

### EXAMPLES

### Example 1: Methods

### Donor Recruitment

Twenty one normal subjects were recruited by posting study information on a University of Washington website seeking healthy subjects. All procedures were approved by the Investigational Review Board and the Radiation Safety Office, respectively, of the University of Washington, School of Medicine.

### Collection and Storage of PLTs

A Terumo, BCT (previously Caridian, BCT) Trima Accel Automated Blood Collection System, using software version 6.0 was used to harvest PLTs for this study according to the manufacturers' guidelines. This produces in-process leukoreduced PLTs. The citrate-to-whole blood ratio used during the apheresis collections was 1:9. After collection, the platelets were stored in the Trima apheresis storage bag. The platelets were stored in a Helmer incubator (Helmer Corporation, Fort Wayne, Ind., USA) at 22±2° C. with constant flatbed agitation at 70 cycles per minute for 8 days. Five ml samples were removed from each donor's apheresis platelets on the day of collection (day 0) and also on days 1, 3, 5, and 8, using standard sterile docking techniques. The samples were aliquoted (unfractionated PLTs in plasma), snap frozen in liquid nitrogen, and stored at −80° C. for subsequent metabolomic analysis. On both day 1 and day 5 post PLT collection, a 2 ml aliquot of the harvested platelets was also obtained to test for white blood cell count, platelet count, mean platelet volume (MPV) (ABX Micros 60, Horiba, Edison, N.J.), pH and blood gases (Bayer 248, Siemans, Malvern, Pa.). Also on both days 1 and 5, an additional 2 ml sample was sent to the University of Washington microbiology laboratory for anaerobic and aerobic culture. On day 5, a platelet sample was sent to the University Microbiology Laboratory for Gram stain. No platelet products were utilized if bacteria were detected by culture or direct Gram stain.

Sampling of PLTs and Radiolabeled Autologous Platelet Recovery and Survival Measurement

On the 5^{th} day of PLT storage, the donor returned for radiolabeling and re-infusion of their stored autologous platelets. A 43-ml aliquot from the apheresis platelets was labeled with ⁵¹Cr, and a separate 50 ml blood sample was drawn from the donor on day 5 to prepare fresh platelets that were labeled with ¹¹¹In. For 3 out of 21 subjects, the isotopes used for the stored and fresh platelets were reversed. Labeling was performed using established techniques⁹. Blood samples were drawn from the donor before, at approximately 2 hours and on days 1, 2, 3, 4 or 5 and 7 or 8 or 9 after transfusion to test for radioactivity bound to platelets using an Auto-Gamma Counter (Model 1480 Automatic Gamma Counter, Wizard 3″, Perkin Elmer. Waltham, Mass., USA). Recoveries were determined by mathematical regression to time point zero to allow an estimation of PLT circulation immediately post-transfusion. Survival was determined by testing the duration (in days) that stored PLTs continued to be detected post-infusion. To control for subject-level differences in baseline PLT function, the recoveries and survivals of the transfused 5-day stored PLTs were expressed as a percentage of their fresh PLT recoveries and survivals, respectively. Platelet recoveries and survivals were calculated using the COST programs¹⁰. Radiolabeled results were red cell elution corrected for all subjects.

### Untargeted and Targeted Metabolomics of PLT Sample

Untargeted metabolomics was carried out by a commercial provider (metabolon inc.)¹¹. New methodologies were developed at BloodworksNW for targeted metabolomic analysis of specific pathways identified by the untargeted approach, including isotopically labeled standards and internal controls that allow precise quantification. A Q-Exactive high resolution/accurate mass spectrometer (Thermo Fisher Scientific. Inc. Waltham. Mass.) using an electrospray ionization (ESI) and Orbitrap mass analyzer was used for untargeted metabolomics. Targeted studies were carried out with an AB Sciex QTrap 6500 with an ESI source coupled with a Waters Acquity I-Class UPLC.

### Statistics

For data display purposes and statistical analysis of untargeted metabolomics, each biochemical was rescaled to set the median equal to 1 by batch and normalized to protein concentration of each sample as measured by the Bradford assay. Any missing values were assumed to be below the limits of detection and these values were imputed with the compound minimum (minimum value imputation). Following median scaling and imputation of missing values, statistical analysis of log-transformed data was performed using R (http://cran.r-project.org/). Pearson correlations and corresponding T-statistics were used to identify biochemicals correlated with platelet storage phenotypes. “Nominal” p values ≤0.05 were considered statistically significant and <0.10 were reported as trends. Multiple comparisons were accounted for by estimating the positive false discovery rate (pFDR) using q values 2. To evaluate the evidence of pathway-level associations, with multiple members identified as significant, a permutation analysis was performed through random permutation of post-transfusion normalized PLT survivals and recoveries. This process was repeated 10,000 times to determine the probability of false conclusion of multiple correlations in the same pathway given a range of significance criteria thresholds (q values and p values).

### Untargeted Metabolomics

Except as indicated in the text below, samples were processed as described previously^{27,28}. For each sample, 100 μL of platelets were used for analyses. Using an automated liquid handler (Hamilton LabStar, Salt Lake City, Utah), protein was precipitated with methanol that contained standards to report on extraction efficiency. The resulting supernatant was split into five aliquots for analysis on the four platforms, with one aliquot retained as a spare. Aliquots, dried under nitrogen and vacuum-desiccated, were subsequently either reconstituted in 50 μL 0.1% formic acid in water (acidic conditions) or in 50 μL 6.5 mM ammonium bicarbonate in water, pH 8 (basic conditions) for the UHPLC/MS/MS analyses or derivatized to a final volume of 50 μL for GC/MS analysis using equal parts bistrimethyl-silyl-trifluoroacetamide and solvent mixture acetonitrile:dichloromethane:cyclohexane (5:4:1) with 5% triethylamine at 60° C. for one hour. In addition, three types of controls were analyzed in concert with the experimental samples: aliquots formed by pooling a small amount of each sample served as technical replicates throughout the data set, extracted water samples served as process blanks, and a cocktail of standards spiked into every analyzed sample allowed instrument performance monitoring. Experimental samples and controls were randomized across three platform run days.

For UHLC/MS/MS analysis, aliquots were separated using a Waters Acquity UPLC (Waters, Millford, Mass.) and analyzed using a Q-Exactive high resolution/accurate mass spectrometer (Thermo Fisher Scientific, Inc., Waltham, Mass.) which consisted of an electrospray ionization (ESI) source and Orbitrap mass analyzer. Derivatized samples for GC/MS were separated on a 5% phenyldimethyl silicone column with helium as the carrier gas and a temperature ramp from 60° C. to 340° C. and then analyzed on a Thermo-Finnigan Trace DSQ MS (Thermo Fisher Scientific, Inc.) operated at unit mass resolving power with electron impact ionization and a 50-750 atomic mass unit scan range.

Metabolites were identified by automated comparison of the ion features in the experimental samples to a reference library of chemical standard entries that included retention time, molecular weight (m/z), preferred adducts, and in-source fragments as well as associated MS spectra, and were curated by visual inspection for quality control using software developed at Metabolon²⁹.

Compounds were identified by comparison to library entries of more than 3500 commercially available purified standards or recurrent unknown entities. The combination of chromatographic properties and mass spectra gave an indication of a match to the specific compound or an isobaric entity. Additional unnamed entities could be identified by virtue of their recurrent nature (both chromatographic and mass spectral). These compounds have the potential to be identified by future acquisition of a matching purified standard or by classical structural analysis.

### Targeted Metabolomics Analysis of Select Pathways

Fatty acid and oxidation product standards and deuterium-labeled internal standards, including prostaglandin E2-d4 (PGE2-d4); arachidonic acid-d8(AA-d8); docosahexaenoic acid-d5 (DHA-d5); eicosapentaenoic acid-d5(EPA-d5); 15S-hydroxy-5Z,8Z,11Z, 13E-eicosatetraenoic acid-d8 (15-HETE-d8); 12S-hydroxy-5Z,8Z,10E, 14Z-eicosatetraenoic acid-d8(12-HETE); 5S-hydroxy-6E,8Z, 11Z,14Z-eicosatetraenoic acid-d8 (5-HETE-d8); 9S-hydroxy-10E, 12Z-octadecadienoic acid-d4 (9-HODE-d4); 13S-hydroxy-9Z,11E-octadecadienoic acid-d4 (13-HODE); (±)9,10-dihydroxy-12Z-octadecenoic acid-d4 (9,10-DiHOME-d4); (±) 12,13-dihydroxy-9Z-octadecenoic acid-d4 (12,13-DiHOME-d4); 9-oxo-10E,12Z-octadecadienoic acid-d3 (9-oxoODE-d3); 13-oxo-9Z, 11E-octadecadienoic acid-d3(13-oxoODE-d3); (±)11,12-dihydroxy-5Z,11Z,14Z-eicosatrienoic acid-d11 (11,12-DiHETrE-d11); Dihomo-γ-Linolenic-d6 (DGLA-d6); Linoleic Acid-d4 (LA-d4); α-Linolenic Acid-d14 (ALA-d14), were purchased from Cayman Chemical (Ann Arbor, Mich.). Isotopically-labeled paraxanthine (¹³C₄, ¹⁵N₃) and caffeine (¹³C₃) were purchased from Sigma-Aldrich (St. Louis, Mo.). Deuterium labeled theobromine (d6) was purchased from Toronto Research Chemicals (Toronto, ON). The following ¹³C, ¹⁵N labeled standards were purchased from Cambridge Isotope Laboratories (Tewksbury, Mass.): 1,3-dimethyluric acid (¹³C₄, ¹⁵N₃); 1,7-dimethyluric acid (¹³C₄, ¹⁵N₃); 3-methyluric acid (¹³C₄, ¹⁵N₃); 7-methyluric acid (¹³C₄, ¹⁵N₃); 1,3,7-trimethyluric acid (¹³C₄, ¹⁵N₃); 7-methylxanthine (¹³C₄, ¹⁵N₃); and theophylline (¹³C₄, ¹⁵N₃). J. T. Baker LC/MS grade acetonitrile and methanol were purchased from VWR (Radnor, Pa.). Optima LC/MS grade 2-propanol was purchased from Fisher Scientific (Waltham, Mass.). MilliQ water was purified in house (18.7MΩ). LC/MS grade ammonium acetate was purchased from Sigma-Aldrich (St. Louis, Mo.).

Fatty acids and oxidized fatty acids were analyzed by liquid chromatography-tandem mass spectrometry with multiple reaction monitoring (LC-MS/MS-MRM). Internal standards were mixed with 100 uL of PLT sample and analytes were extracted using methanol. The supernatant was diluted 1:1 (v/v) with 10 mM ammonium acetate in water for LC-MS analysis of lipids. LC-MS/MS analysis was performed using an AB Sciex QTrap 6500 with an ESI source coupled with a Waters Acquity I-Class UPLC. Analytes were separated on HSS T3 column (2.1×100 mm, 1.8 μm, Waters) and detected by MS/MS detector using multiple reaction monitoring (MRM) in the negative ion mode. Analytes were quantified relative to their isotopically-labeled analogues except for those indicated with a numeral superscript in the table.

Caffeine and its metabolites were also analyzed by LC-MS/MS-MRM. Internal standards were mixed with 50 uL of PLT sample and analytes were extracted using methanol. The supernatant was diluted 1:8 (v/v) with 0.1% formic acid in water for LC-MS analysis. LC-MS/MS analysis was performed using an AB Sciex QTrap 6500 with an ESI source coupled with a Waters Acquity I-Class UPLC. Analytes were separated on Cortecs UPLC C18 column (2.1×100 mm, 1.6 μm. Waters) and detected by MS/MS detector using multiple reaction monitoring (MRM) in the positive ion mode. Analytes were quantified relative to their isotopically-labeled analogues except for those indicated with a numeral superscript in the table.

### Example 2: In Vivo Radiolabeled Recoveries and Survivals of Autologous Platelets

Absolute recoveries and survivals of fresh and stored PLTs are shown in Table 1 and normalized PLT recoveries were calculated by correcting stored values with fresh values. As commonly observed in the field, there was only a weak correlation between normalized PLT recoveries and survivals of stored PLTs within a given donor (Pearson's=0.018). No significant correlations were observed between normalized PLT recoveries or survivals and age or gender of donors, nor in measurements of the 5-day stored apheresis PLTs (i.e. MPV. Average PLT count, or pH of the stored PLTs). No unit of PLTs had a pH of less than 7.08.

### Example 3: Biochemicals Identified

In this study, 1136 distinct biochemicals were identified (740 compounds of known structural identity and 396 unnamed compounds). With a traditional “nominal” p value cut off of 0.05, it is estimated that approximately 57 compounds should correlate with a given phenotype (i. e. PLT survival or recovery), just by chance alone. Thus, when considering results, we report q values in addition to p values, as described in Example 1.

### Example 4: Metabolites and Metabolic Pathways Associated with 24-Hr Recoveries of Transfused PLTs

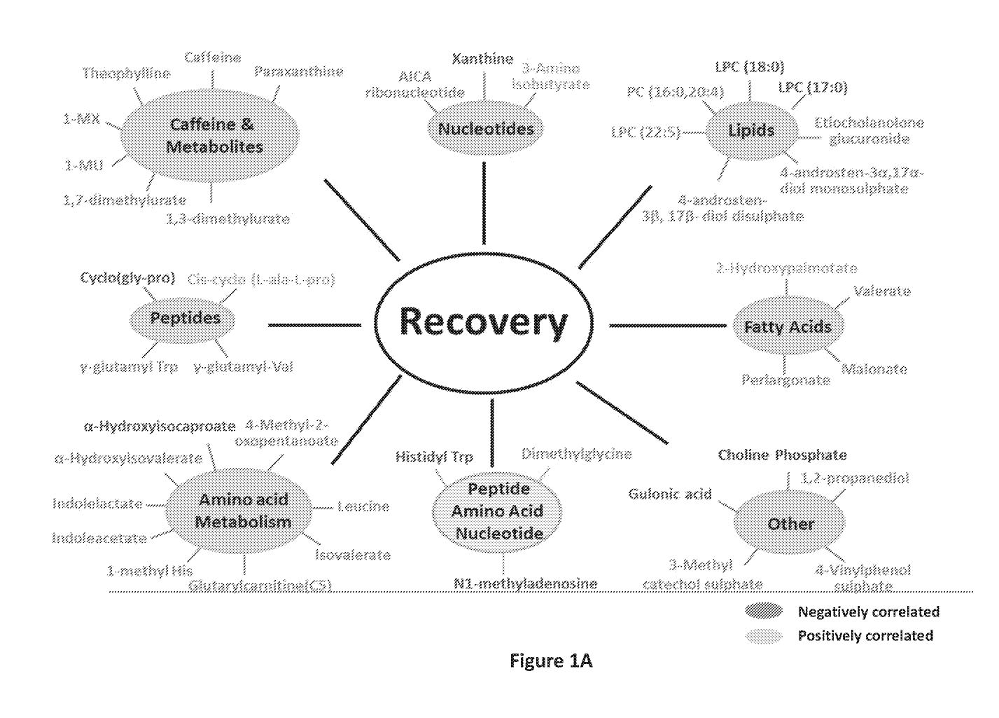

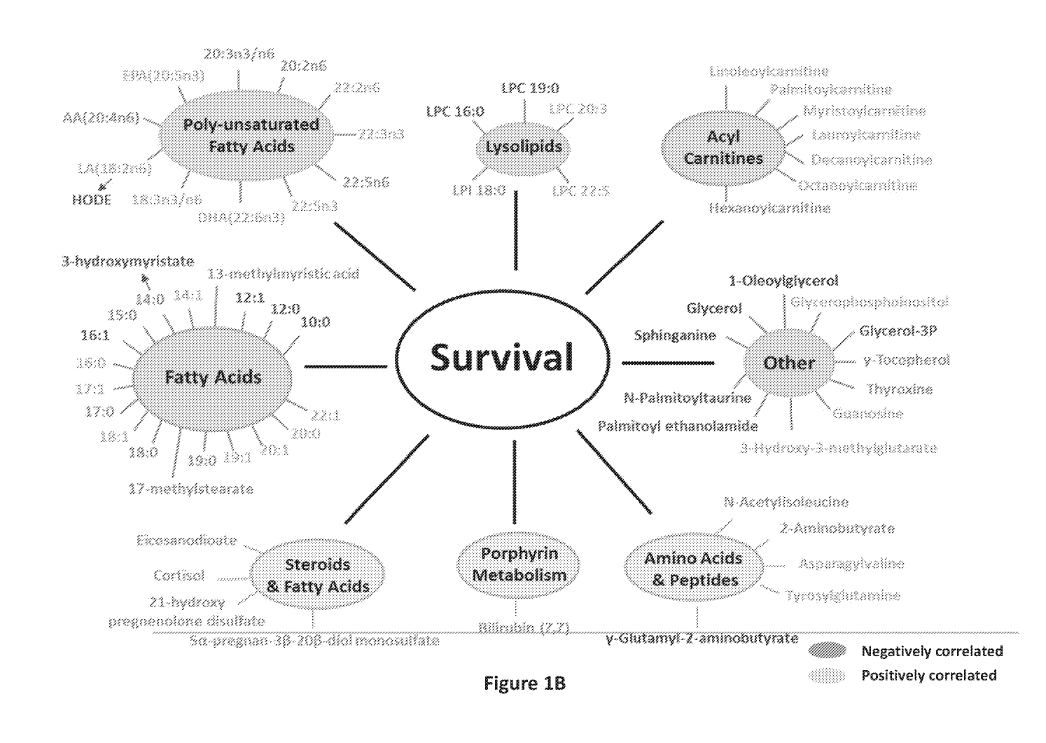

Linear correlations of individual metabolite levels on the day of infusion (day 5 of storage) with normalized PLT recoveries were evaluated by Pearson correlation coefficients, and the results are summarized in Table 4. A schematic view of identified classes of compounds and individual metabolites is presented in FIG. 1A. Using a cutoff of p<0.05 as statistically significant, only 3 metabolites had a positive correlation with PLT recoveries, each belonging to separate metabolic pathways (histidyltryptophan, dimethylglycine, and N1-methyladenosine). In contrast, 48 metabolites had a negative correlation of statistical significance. If we take as discoveries (to be evaluated in future targeted studies) all 51 metabolites satisfying nominal significance (with p<0.05, listed in Table 4), the maximum q value of 0.68 suggests that 35 (approximately equal to 68%) of these correlations may be expected to represent “false discoveries” observed by chance after accounting for multiple hypothesis tests, leading to the conclusion that an estimated 16 of these metabolites represent true correlations in the population represented by this study. The smallest q value observed in this set is 0.3, suggesting that the estimated risk that the corresponding association was observed by chance is 30%. In addition to reporting individual correlations, we noted that many of the negatively correlated metabolites were related metabolites within a given pathway, leading to post-hoc pathway significance evaluation discussed in a later section.

Analysis of lipids as a general class demonstrated the presence of 5 steroid hormones, each within the known pathways of androgen/estrogen metabolism, which correlated negatively with normalized PLT recoveries. Such steroid hormones are known to be active signaling molecules for androgen receptors. Additional lipid species from isolated pathways were also detected, including short and medium chain fatty acids, a monohydroxy fatty acid, and lipids associated with phospholipid metabolism and fatty acid synthesis; however, these additional species were not members of a common pathway.

A series of xenobiotic metabolites were detected, which are the metabolic breakdown products of starting materials derived from xenogenous sources. Of particular note are caffeine and its metabolites, which are classified as members of Xanthine metabolism (Table 4). Nine separate metabolites each correlated negatively with PLT recoveries (including caffeine itself). Each of these metabolites was detectable at the day of collection, and levels did not change significantly over the course of storage. Together, these data indicate that the presence of caffeine metabolites in collected PLTs were inversely correlated with PLT recoveries.

Additional clustering of components of metabolic pathways that correlate negatively with PLT recoveries include 5 members of amino acid metabolism (Leucine, Isoleucine and Valine), and also 3 members of Tryptophan metabolism. Individual metabolites of lysine and histidine were also detected, as were two dipeptide species (cyclo(gly-pro) and cis-Cyclo[L-ala-LPro]). Two gamma-glutamyl amino acids were identified, which can be involved in glutathione metabolism and/or xenobiotic detoxification. In the context of nucleotide metabolism, two purine metabolites (AICA ribonucleotide and xanthine) and one pyrimidine metabolite (3-aminoisobutyrate) were identified. Finally, gulonic acid, a metabolite of ascorbate/adarate was identified.

### Example 5: Metabolites and Metabolic Pathways Associated with Survival of Transfused PLTs

The correlation of individual metabolites detected on the day of infusion (day 5 of storage) was calculated as a function of normalized PLT survivals using Pearson correlation coefficients, and the results are summarized in Table 5. A schematic view of identified classes of compounds and individual metabolites is presented in FIG. 1B.

A total of 69 metabolites satisfying nominal significance (p<0.05) were identified. The maximum q value of 0.63 suggests that 44 of the 69 identified correlations may be expected to represent false discoveries, and corresponding to an estimated 25 true discoveries. The smallest q value observed in this set is 0.1878, with three out of four metabolites with this value estimated to represent true correlations not identified based on chance alone.

Twelve analytes had significant positive correlations with PLT survival (Table 5). Of particular interest was the clustering of steroid hormones, including cortisol and pregnclone derivatives. Of note, whereas pregnenolone derivatives correlated positively with PLT survival it is mostly androgen derivatives that correlated negatively with PLT recoveries. It is also worth noting that two dicarboxylic fatty acids (3-methyladipate and eicosanodioate) correlated positively with PLT survivals.

Fifty-nine metabolites had a negative correlation with PLT survivals, including clustering to several metabolic pathways (Table 5). In particular, lipid metabolism emerged as a main determinant in this regard. First, 7 acyl camitine fatty acids had a significant inverse correlation with PLT survival. In general, acyl carnitine fatty acids are involved in beta-oxidation of fatty acids in the process of generating acetyl-coA to feed into the citric acid cycle. However, as greater levels of acyl carnitine fatty acids are seen with reduced PLT survivals, it suggests a negative effect of beta oxidation of fatty acids. Second, 6 lysophospholipids were also inversely correlated with PLT survival. Of note, 5 out of the 6 lysophospholipids were derived from choline species, with ⅙ derived from inositol. Glycerophosphoinositol also was inversely correlated. In addition, a number of the byproducts of lipid metabolism and breakdown were inversely correlated with PLT survivals, including glycerol, glycerol-3-phosphate, 1-monolein, and sphinganine.

A substantial number of free fatty acids had a negative correlation with PLT survivals. First, 15 separate long chain fatty acids had a significant negative correlation with PLT survivals. The chain length ranged from 14 carbons (myristoleate) to 22 carbons (erucate), and included both saturated and unsaturated fatty acids. Second, 3 shorter length (10-12 carbons) fatty acids had a significant negative correlation. Third, 11 polyunsaturated fatty acids had a significant negative correlation, including a number of molecules known to be active in signaling and/or biological precursors of bioactive lipids (e.g. Arachidonic acid (AA), docosahexaenoic acid (DHA), eicosapentaenoic acid (EPA), and linoleic acid (LA)). Additional fatty acids with significant inverse correlations included 2 branched fatty acids and 2 monohydroxy fatty acids.

Other isolated compounds with significant inverse correlations included molecules involved in mevalonate metabolism, purine metabolism, endocannabinoids, phenylalanine and tyrosine metabolism and also gamma-tocopherol (a form of vitamin E).

### Example 6: Statistical Consideration of Multiple Components of a Common Pathway

As discussed above, in some cases multiple metabolites in a common pathway were correlated with PLT function; neither the traditional p value nor the pFDR q value approach consider the evidence for pathway-level correlations. In the permutation analysis of pathway level associations, using individual metabolite significance criteria of p value <0.05 and q value <0.3, for correlation with survival, 6 or more metabolites common to a single pathway were required to limit the false positive rate of pathway identification below 5%; for correlation with recovery, identification of 6 metabolites corresponded to a false positive rate of 5.3%, just above the traditional 5% threshold. The q-value threshold of 30% for individual metabolites was specified as a maximal acceptable individual metabolite error threshold to be applied to evaluate the results for corresponding pathway associations in this study. A stricter criteria for q value of 20% would reduce the false positive error, while also reducing the power to detect true pathway associations (data not shown). Long chain fatty acids satisfied the former requirement (8 metabolites with q<0.3 among 15 with p<0.05). Polyunsaturated fatty acids (4 metabolites with q<0.3 among 12 p<0.05) and caffeine (2 metabolitics with q<0.3 among 9 with p<0.05) had pathway false positive rates of 6.8% and 15% respectively. Acylcamitines (4 metabolites with q<0.3 among 7 with p<0.05) also had a pathway false positive rate of 6.8%. Though 6.8% and 15% false positive error rates fall above the traditional threshold for significance, these errors were within the range often considered acceptable for generating hypotheses for future exploration in targeted studies.

### Example 7: Targeted Analysis of Caffeine and its Metabolites; Concentrations and Correlations

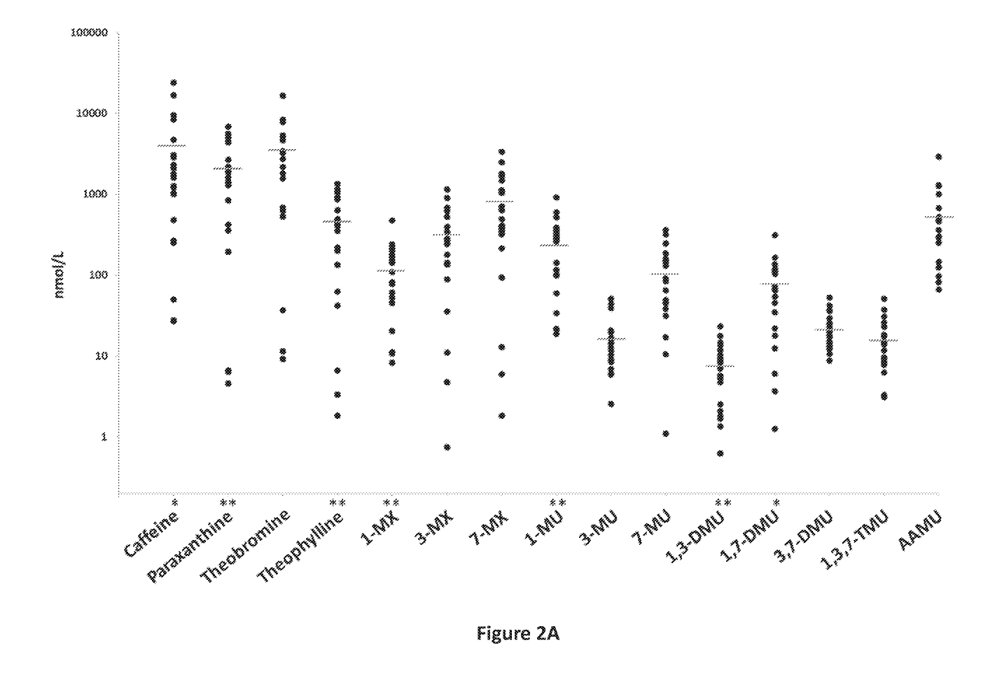

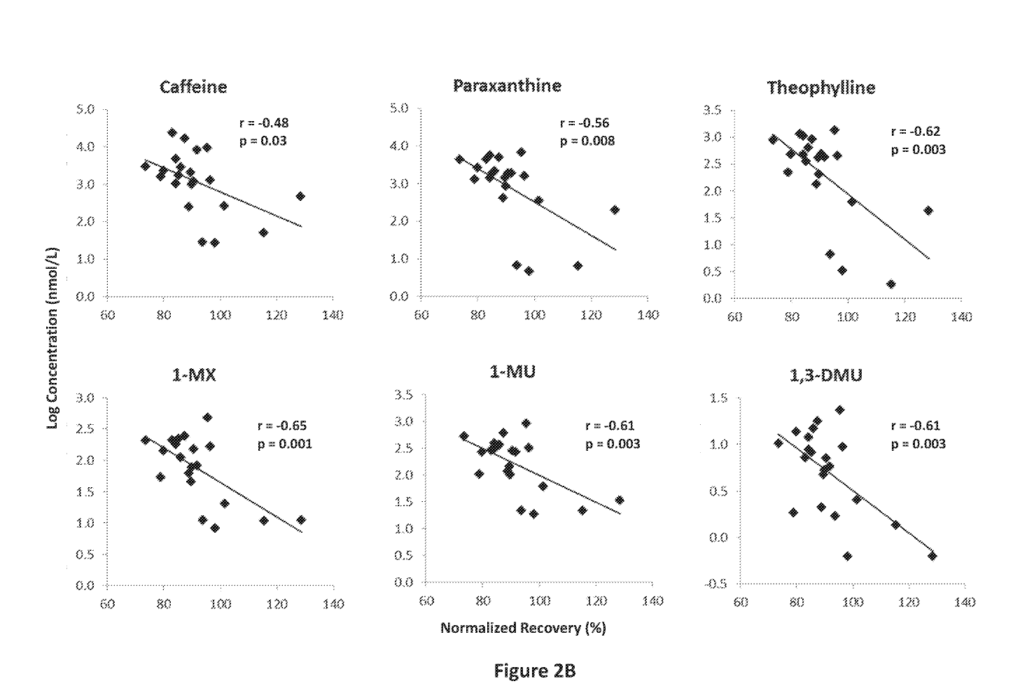

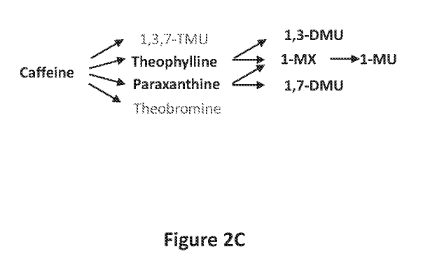

Based upon the above findings, a targeted metabolomic assay was developed to provide precise quantitation of caffeine and its metabolites. Fourteen distinct known metabolites of caffeine were quantified in donor PLT units (increased from the 9 metabolites identified by the untargeted approach), with levels varying from nanomolar to micromolar concentration in an individual PLT product (FIG. 2A). Consistent with the untargeted analysis, there was a significant negative correlation of caffeine and 6 of its metabolites, all with p values below 0.05 (in most cases less than 0.01) (FIG. 2B). However, no significant correlation was seen with other metabolites of caffeine. Of note, metabolites formed from conversion of caffeine to theophylline and paraxanthine, but not of the theobromine pathway correlated negatively with PLT recoveries (FIG. 1C).

### Example 8: Targeted Quantitation of Products of Lipid Oxidation and their Precursors

A targeted metabolomic method was also developed for products of lipid oxidation and their precursors (see methods). Targeted species included common polyunsaturated fatty acids: AA, DHA, EPA, and LA. Likewise, a panel of oxidized products of these precursors was measured. 20 out of 27 fatty acids analyzed had a statistically significant (p value <0.05) negative correlation with PLT survival, 11 of which had a p value <0.01. An additional 7 had a “trend” for a negative correlation (p values between 0.5 and 0.1) (Table 2). In contrast, only 1 of the 15 oxidized fatty acids had a significant correlation to recovery (9(10)-EpOME) (Table 3; p value 0.026). Also consistent with untargeted findings, 9 out of 15 products of lipid oxidation measured had statistically significant inverse correlations with PLT survival post-transfusion (Table 3). Thus, the targeted data on lipid species verified the negative correlation that had been detected by untargeted metabolomics in these samples (Tables 2 and 3). In addition, many new products of oxidation that were included in the targeted panel (Table 3), which were not detected by untargeted metabolomics (Tables 4 and 5) showed a significant inverse correlation, extending the observation as well as verifying it.

It is also worth noting the quantitation of fatty acids and their oxidized products in PLTs stored for 5 days. Predicting PLT circulation post-transfusion notwithstanding, a number of the identified lipids have known potent biological activities well within the concentrations detected. For example, arachidonic acid concentrations ranged from 2.9-12.3 micromolar and 12-HETE ranged from 48.8-813 nanomolar. It has been shown that arachidonic acid and 12-HETE have direct physiological effects at these concentrations al

### Example 9: Application of the Above Biochemical Mediators as a Diagnostic Test of PLT Analysis

The above biochemical mediators of PLT unit quality may be applied to the evaluation of PLT units in several different ways. First, a sample of a PLT unit can be subjected to mass spectrometry and the profile of the above biochemical mediators can be generated (all from a single sample). This profile would then be used to predict the post-transfusion survival of a PLT unit. Such information would allow 3 distinct medical advantages: 1) direction of better units of PLTs to patients whose disease status makes them particularly susceptible to bleeding from thrombocytopenia, 2) management of the blood supply such that donors with good storage properties can be preferentially recruited, 3) decrease the number of units any given patient receives, thereby decreasing exposure to multiple donors and deceasing risk of both allommunization and infectious disease transmission, and 4) identification of PLT units with lower leukotrienes and prostaglandins, thus allowing units with higher amounts of such substances to not be transfused into patients predicted to be sensitive to such substances. Alternatively, individual assays could be run on a much smaller platform by traditional assay techniques (i.e. ELISA, enzymatic assay, etc.). Such would allow a simplified platform with a less expensive instrumentation. For such purposes, a small number of the above chemical entities that were representative of the whole would be identified and measured.

### Discussion

Two previous studies have carried out metabolomics analysis of stored PLTs, focusing both on a general characterization of the PLT storage lesion, and also on a juxtaposition of differences in PLT metabolism under distinct storage conditions L. These studies represent important steps forward in our empirical understanding of what occurs to PLTs when they are stored. However, to the best of our knowledge, the current work is the first study to include in vivo performance of the PLTs after transfusion, thus generating the ability to correlate changes in a given metabolite (or metabolic pathway) with post-transfusion PLT viability. As suggested by Nemkov et al, study design to allow such correlations is a necessary step in omics based transfusion research L.

The majority of the data presented was analyzed on day 5, as this was both the day in vivo studies were carried out, and also because PLT storage has traditionally been limited to 5 days. However, the change of metabolites over time is an issue of considerable importance, as shown in FIG. 1 and Tables 2 and 3. There were no metabolites that correlated with PLT survival, which had p values less than 0.05 and q values less than 0.3 at time of collection (data not shown). However, in the case of recoveries, the same clustering of caffeine metabolites was present at time of collection, with 8 metabolites having a p value of less than 0.05, but all with high q values (data not shown). Likewise, the same steroid hormones detected in day 5 samples had correlated with p values less than 0.05 at time of collection, but had very high q values. Thus, it remains possible that metabolites present at time of collection may be predictive of how PLTs store, but they are fewer in number than those that emerge over the storage time course, and if they exist, the current study was unable to detect them with statistical significance.

Levels of caffeine and its metabolites were stable from the time of collection throughout the observed storage period; thus it appears that caffeine metabolism is not ongoing during PLT storage. In this study, correlation between caffeine metabolites and recovery reached nominal significance. This did not achieve statistical trend status for the untargeted study, but was verified by targeted analyses. The effect of caffeine consumption on function of platelets is controversial, with some investigators reporting an effect and others not¹⁸⁻²¹; however, to the best of our knowledge, only one study has reported that addition of caffeine to PLTs had effects that may be relevant to PLT storage²². It is unclear from the current data if caffeine has a direct effect itself, if metabolites of caffeine have an effect, or if caffeine is simply associated with other factors that affect PLT biology. Formal controlled studies surrounding caffeine consumption must be performed to test the above hypotheses.

The targeted analysis of free fatty acids and their oxidative metabolites provided precise quantitation of this class of compounds, and demonstrated a statistically significant negative correlation between multiple analytes and PLT survivals (see Tables 2 and 3). Because this was the same cohort as analyzed in the untargeted metabolomics, the correlation does not provide independent verification, and analysis of an independent cohort is required to assess the extent to which results from this study may generalize to the broader donor population.

In addition to correlating with PLT survivals, the accumulation of FAs and their oxidative products have potential direct sequelae upon recipient biology as a result of their infusions. It has been shown that AAs and HETEs, which accumulate in PLT storage, are capable of priming neutrophils and causing acute lung injury in a rat model of TRALI²³. AA accumulated to high levels in many of the stored PLT units, achieving micromolar concentrations in many cases. Infusion of as little as 1 nmol of AA had a demonstrable effect upon human volunteers, largely through metabolism by the recipient into cicosanoids²⁴. Because the average unit of stored PLTs had 4.8 micromolar AA, this was approximately 1000 times the dose that caused an effect in humans²⁴. Likewise, cutaneous injection of 12-HETE into human volunteers causes neutrophil infiltrates at a dose of 200 ng^{13,14}. Finally, since DHA and EPA metabolism can lead to anti-inflammatory products, and both DHA and EPA also accumulate in PLT units, mutually antagonistic pathways may be simultaneously at play.

To the best of our knowledge, this is the first work to provide a detailed metabolomics analysis linked to in vivo biology of the transfused product. In addition, these findings should be used in the context of recent progress by Schubert et al. in correlating proteomic changes of PLT storage with post-transfusion circulation^{25,26}. This approach identified a number of candidate analytes, which will require subsequent studies on additional cohorts to determine the reproducibility of these findings. Nevertheless, the utility of these findings extends to a greater understanding of which components of the PLT storage lesion are functionally important. Because the analyzed samples were unfractionated PLTs in plasma, consideration should be given to the fact that both PLT and plasma metabolites are included in the data set. If validated, these metrics may serve as a rapid in vitro measure to guide development of new PLT storage solutions. Of direct medical relevance to sequelae of PLT transfusion is the accumulation of certain fatty acids and their oxidation products. Given the relatively high level of bioactive lipid accumulation, which well exceeds the doses known to have physiological effects, these studies suggest a rational basis to screening PLT units for cicosanoids and preferentially using PLT units with lower cicosanoids in patients at risk of ALI.

### REFERENCES

1. Thiagarajan P, Afshar-Kharghan V. Platelet transfusion therapy. Hematol Oncol Clin North Am 2013; 27: 629-43.
2. Hess J R. Resuscitation of trauma-induced coagulopathy. Hematology Am Soc Hematol Educ Program 2013; 2013: 664-7.
3. Cardigan R, Turner C, Harrison P. Current methods of assessing platelet function; relevance to transfusion medicine. Vox Sang 2005; 88: 153-63.
4. Dumont L J, AuBuchon J P, Gulliksson H, Slichter S J, Elfath M D, Holme S, Murphy J R, Rose L E, Popovsky M A, Murphy S. In vitro pH effects on in vivo recovery and survival of platelets: an analysis by the BEST Collaborative. Transfusion 2006; 46: 1300-5.
5. Holme S, Sweeney J D, Sawyer S, Elfath M D. The expression of p-selectin during collection, processing, and storage of platelet concentrates: relationship to loss of in vivo viability. Transfusion 1997; 37: 12-7.
6. Mitrovic S, Mitrovic D, Dragojlovic Z, Todorovic V. Cuk G. Nedeljkovic N, Gligorovic V. A simple morphological score for the quality control of platelet concentrates. Haematologia (Budap) 1997; 28: 239-46.
7. Leytin V, Allen D J, Gwozdz A, Garvey B, Freedman J. Role of platclet surface glycoprotein Ibalpha and P-selectin in the clearance of transfused platelet concentrates. Transfusion 2004; 44: 1487-95.
8. Dumont Li, AuBuchon J P, Whitley P, Herschel L H, Johnson A, McNeil D, Sawyer S. Roger J C. Seven-day storage of single-donor platelets: recovery and survival in an autologous transfusion study. Transfusion 2002; 42: 847-54.
9. Snyder E L, MoroffG. Simon T, Heaton A. Recommended methods for conducting radiolabeled platelet survival studies. Transfusion 1986; 26: 37-42.
10. Lotter M G. Rabe W L, Van Zyl J M, Heyns A D, Wessels P, Kotze H F, Minnaar P C. A computer program in compiled BASIC for the IBM personal computer to calculate the mean platelet survival time with the multiple-hit and weighted mean methods. Comput Biol Med 1988; 18: 305-15.
11. Evans A M, Bridgewater B R, Liu Q. Mitchell M W, Robinson R J, Dai H, Stewart S J, DeHaven C D, Miller L A D. High resolution mass spectrometry improves data quantity and quality as compared to unit mass resolution mass spectrometry in high-throughput profiling metabolomics. Metabolomics 2014; 4: 132.
12. Storey J D, Tibshirani R. Statistical significance for genomewide studies. Proc Natl Acad Sci USA 2003; 100: 9440-5.
13. Dowd P M, Greaves M W. Cutaneous responses to lipoxygenase products of arachidonic acid. Acta Derm Venereol Suppl (Stockh) 1985; 120: 18-22.
14. Dowd P M, Kobza Black A. Woollard P M, Camp R D, Greaves M W. Cutaneous responses to 12-hydroxy-5,8,10,14-eicosatetraenoic acid (12-HETE). J Invest Dermatol 1985; 84: 537-41.
15. Paglia G, Sigurjonsson O E, Rolfsson O, Hansen M B, Brynjolfsson S, Gudmundsson S, Palsson B O. Metabolomic analysis of platelets during storage: a comparison between apheresis- and buffy coat-derived platelet concentrates. Transfusion 2015; 55: 301-13.
16. Paglia G, Sigurjonsson O E, Rolfsson O, Valgeirsdottir S. Hansen M B, Brynjolfsson S, Gudmundsson S, Palsson B O. Comprehensive metabolomic study of platelets reveals the expression of discrete metabolic phenotypes during storage. Transfusion 2014; 54: 2911-23.
17. Nemkov T, Hansen K C. Dumont L J, D'Alessandro A. Metabolomics in transfusion medicine. Transfusion 2015.
18. Biaggioni I, Paul S, Puckett A, Arzubiaga C. Caffeine and theophylline as adenosine receptor antagonists in humans. J Pharmacol Exp Ther 1991; 258: 588-93.
19. Cavalcante J W, Santos P R, Jr., Menezes M G, Marques H O, Cavalcante L P, Pacheco W S. Influence of caffeine on blood pressure and platelet aggregation. Arq Bras Cardiol 2000; 75: 97-105.
20. Fredholm B B. Astra Award Lecture. Adenosine, adenosine receptors and the actions of caffeine. Pharmacol Toxicol 1995; 76: 93-101.
21. Varani K, Portaluppi F, Merighi S. Ongini E, Belardinelli L, Borea P A. Caffeine alters A2A adenosine receptors and their function in human platelets. Circulation 1999; 99: 2499-502.
22. Bode A P, Holme S. Heaton W A, Swanson M S. Sustained elevation of intracellular cyclic 3′-5′ adenosine monophosphate is necessary for preservation of platelet integrity during long-term storage at 22 degrees C. Blood 1994; 83: 1235-43.
23. Silliman C C, Bjornsen A J, Wyman T H I Kelher M, Allard J, Bieber S, Voelkel N F. Plasma and lipids from stored platelets cause acute lung injury in an animal model. Transfusion 2003; 43: 633-40.
24. Bhagat K, Collier J, Vallance P. Vasodilatation to arachidonic acid in humans. An insight into endogenous prostanoids and effects of aspirin. Circulation 1995; 92: 2113-8.
25. Schubert P, Culibrk B, Karwal S, Slichter S J, Devine D V. Optimization of platelet concentrate quality: application of proteomic technologies to donor management. J Proteomics 2012; 76 Spec No.: 329-36.
26. Devine D V, Schubert P. Proteomic applications in blood transfusion: working the jigsaw puzzle. Vox Sang 2011, 100: 84-91.
27. Evans A M, DeHaven C D, Barrett T, Mitchell M. Milgram E. Integrated, nontargeted ultrahigh performance liquid chromatography/electrospray ionization tandem mass spectrometry platform for the identification and relative quantification of the small-molecule complement of biological systems. Anal Chem. 2009; 81(16):6656-6667.
28. Ohta T, Masutomi N, Tsutsui N. et al. Untargeted metabolomic profiling as an evaluative tool of fenofibrate-induced toxicology in Fischer 344 male rats. Toxicol Pathol. 2009; 37(4):521-535.
29. Dehaven C D, Evans A M, Dai H. Lawton K A. Organization of GC/MS and LC/MS metabolomics data into chemical libraries. J Cheminform. 2010; 2(1):9.

TABLE 1
Platelet survival and recovery

| | 24-hr | 24-hr | Normalized | | | Normalized |
|---|---|---|---|---|---|---|
| | Recovery of | Recovery of | 24-hr | Survival | Survival | Survival of |
| | Fresh PLTs | Stored PLTs | Recovery | Fresh PLTs | Stored PLTs | Stored PLTs |
| Subject | (%) | (%) | (% of Fresh) | (Days) | (Days) | (% of Fresh) |
| 1 | 87 | 78 | 90 | 7.9 | 5.3 | 67 |
| 2 | 63 | 57 | 91 | 8.4 | 5.7 | 68 |
| 3 | 65 | 75 | 115 | 6.2 | 3.4 | 55 |
| 4 | 52 | 51 | 98 | 10.3 | 8.8 | 85 |
| 5 | 45 | 36 | 80 | 7.4 | 5.9 | 80 |
| 6 | 57 | 55 | 97 | 7.9 | 5.7 | 72 |
| 7 | 65 | 54 | 83 | 8.3 | 7.0 | 84 |
| 8 | 47 | 60 | 129 | 8.3 | 6.4 | 77 |
| 9 | 44 | 42 | 96 | 8.0 | 7.4 | 93 |
| 10 | 54 | 46 | 85 | 8.4 | 6.3 | 75 |
| 11 | 54 | 48 | 89 | 7.8 | 6.6 | 85 |
| 12 | 79 | 74 | 94 | 9.0 | 7.0 | 78 |
| 13 | 64 | 54 | 84 | 8.5 | 5.3 | 62 |
| 14 | 52 | 41 | 79 | 10.6 | 8.2 | 77 |
| 15 | 62 | 57 | 92 | 8.5 | 5.8 | 68 |
| 16 | 70 | 59 | 84 | 9.6 | 7.8 | 81 |
| 17 | 62 | 63 | 102 | 8.4 | 5.3 | 63 |
| 18 | 64 | 55 | 86 | 9.4 | 7.0 | 75 |
| 19 | 59 | 53 | 90 | 8.7 | 6.8 | 78 |
| 20 | 56 | 49 | 88 | 9.4 | 7.5 | 80 |
| 21 | 83 | 61 | 74 | 9.0 | 3.8 | 42 |
| Average ± SD | 61 ± 12 | 56 ± 11 | 92 ± 12 | 8.6 ± 1 | 6.3 ± 1.3 | 74 ± 11 |

TABLE 2
Concentration of fatty acids and correlation to survival and recovery of PLT at day 5

| | Conc (day 0) | Conc (day 5) | Range | Correlation to | | Correlation to | |
|---|---|---|---|---|---|---|---|
| | Mean ± SD | Mean ± SD | (day 5) | Survival^{a} | | Recovery^{b} | |
| Analytes | (μmol/L) | (μmol/L) | (μmol/L) | r | p value | r | p value |
| AA (20:4n6)* | 4.9 ± 2.2 | 6.4 ± 3.0 | 3.6-16.2 | −0.44 | 0.044 | −0.14 | 0.543 |
| DHA(22:6n3)* | 1.4 ± 1.2 | 2.3 ± 1.5 | 0.7-6.0 | −0.45 | 0.039 | 0.13 | 0.572 |
| EPA(20:5n3) | 0.24 ± 0.16 | 0.31 ± 0.19 | 0.06-0.7 | −0.38 | 0.093 | 0.09 | 0.682 |
| LA(18:2n6)** | 20.5 ± 16.9 | 37.6 ± 24.0 | 11.6-91.4 | −0.56 | 0.008 | 0.15 | 0.504 |
| ALA(18:3n3) | 3.9 ± 4.4 | 5.5 ± 5.4 | 0.8-22.8 | −0.39 | 0.084 | 0.05 | 0.829 |
| DGLA(20:3n6)** | 0.50 ± 0.27 | 0.48 ± 0.20 | 0.2-1.0 | −0.60 | 0.004 | −0.08 | 0.729 |
| ²Capric acid (10:0) | 1.4 ± 0.7 | 3.1 ± 2.0 | 1.1-10.4 | −0.42 | 0.061 | 0.14 | 0.539 |
| ²Lauric acid (12:0)** | 4.4 ± 5.7 | 11.5 ± 13.2 | 2.4-55.0 | −0.65 | 0.002 | 0.11 | 0.626 |
| ²Dodecenoic acid (12:1)* | 0.29 ± 0.33 | 1.15 ± 0.63 | 0.4-2.5 | −0.45 | 0.039 | −0.01 | 0.954 |
| ¹Myristic acid (14:0)** | 7.6 ± 4.1 | 10.0 ± 13.5 | 2.8-64.6 | −0.55 | 0.009 | −0.04 | 0.857 |
| ¹Myristoleic acid (14:1) | 0.57 ± 0.75 | 1.2 ± 1.6 | 0.1-7.4 | −0.42 | 0.059 | −0.05 | 0.817 |
| ¹Pentadecanoic acid (15:0)* | 1.0 ± 0.36 | 1.1 ± 1.1 | 0.4-5.2 | −0.50 | 0.020 | −0.06 | 0.790 |
| ¹Palmitic acid (16:0)** | 70.9 ± 32.9 | 89.1 ± 71.2 | 42.9-362 | −0.56 | 0.009 | −0.09 | 0.682 |
| ¹Palmitoleic acid (16:1)** | 6.1 ± 7.47 | 11.2 ± 16.1 | 1.5-76.2 | −0.59 | 0.005 | −0.03 | 0.896 |
| ¹Margaric acid (17:0) | 1.9 ± 0.6 | 1.9 ± 1.5 | 0.8-6.9 | −0.43 | 0.052 | −0.19 | 0.400 |
| ¹Heptadecenoic acid (17:1)** | 0.53 ± 0.50 | 0.97 ± 1.1 | 0.2-5.1 | −0.57 | 0.006 | −0.01 | 0.981 |
| ¹Stearic acid (18:0) | 47.7 ± 13.6 | 38.7 ± 26.7 | 13.5-116 | −0.43 | 0.053 | −0.21 | 0.360 |
| Oleic acid (18:1)* | 81.9 ± 67.8 | 116 ± 84 | 30.1-390 | −0.51 | 0.018 | 0.06 | 0.806 |
| ¹Nonadecanoic acid (19:0) | 0.15 ± 0.05 | 0.12 ± 0.09 | 0.03-0.39 | −0.35 | 0.119 | −0.13 | 0.563 |
| ¹Nonadecenoic acid (19:1)* | 0.25 ± 0.20 | 0.28 ± 0.20 | 0.09-0.94 | −0.51 | 0.018 | −0.02 | 0.944 |
| ¹Eicosenoic acid (20:1)** | 0.86 ± 0.68 | 0.95 ± 0.63 | 0.37-3.03 | −0.57 | 0.007 | 0.04 | 0.873 |
| ¹Eicosadienoic acid (20:2)** | 0.56 ± 0.49 | 0.41 ± 0.22 | 0.16-1.04 | −0.58 | 0.006 | 0.09 | 0.711 |
| ¹Erucic acid (22:1)** | 0.13 ± 0.05 | 0.15 ± 0.11 | 0.05-0.50 | −0.55 | 0.010 | 0.05 | 0.846 |
| ¹Adrenic Acid (22:4n6)* | 0.34 ± 0.26 | 0.37 ± 0.26 | 0.2-1.1 | −0.53 | 0.014 | −0.19 | 0.40 |
| ¹Docosapentaenoic acid (22:5n3) | 0.39 ± 0.32 | 0.44 ± 0.30 | 0.2-1.4 | −0.66 | 0.001 | −0.12 | 0.619 |

Analytes were quantified by isotopically labeled analogues except for
¹16:0-d3,
²12:0-d3
^{a}Correlation of log concentration at day 5 to normalized survival
^{b}Correlation of log concentration at day 5 to normalized recovery
*p < 0.05 and
**p < 0.01 by Pearson correlation to normalized survival

TABLE 3
Concentration of oxidized fatty acids and correlation to
survival and recovery of PLT at days 0 and 5 of Storage

| | Conc (day 0) | | Range | Correlation to | | Correlation to | |
|---|---|---|---|---|---|---|---|
| | Mean ± SD | Conc (day 5) | (day 5) | Survival^{a} | | Recovery^{b} | |
| Analytes | (nmol/L) | Mean ± SD | (nmol/L) | r | p value | r | p value |
| 12-HETE * | 80.9 ± 77.4 | 183 ± 170 | 48.3-813 | −0.47 | 0.031 | −0.18 | 0.432 |
| 15-HETE * | 1.2 ± 0.7 | 4.6 ± 1.5 | 2.3-8.4 | −0.50 | 0.022 | −0.28 | 0.212 |
| ¹11-HETE * | 3.4 ± 3.0 | 8.7 ± 6.8 | 3.1-32.3 | −0.52 | 0.015 | −0.28 | 0.226 |
| ¹8-HETE | 1.6 ± 1.2 | 2.9 ± 1.6 | 0.6-7.4 | −0.30 | 0.192 | −0.09 | 0.694 |
| ¹12-HEPE * | 16.4 ± 23.6 | 29.8 ± 40.0 | 5.2-194 | −0.59 | 0.004 | −0.14 | 0.552 |
| ¹14-HDoHE ** | 47.5 ± 72.3 | 130 ± 112 | 39.1-533 | −0.56 | 0.008 | 0.04 | 0.874 |
| 9-HODE ** | 20.4 ± 21.3 | 25.1 ± 47.9 | 4.2-204.6 | −0.62 | 0.003 | 0.02 | 0.916 |
| 13-HODE * | 37.5 ± 34.3 | 94.2 ± 112 | 30.4-430 | −0.55 | 0.010 | −0.03 | 0.882 |
| 9,10-DiHOME * | 42.1 ± 32.8 | 136 ± 9.1 | 55.9-372 | −0.53 | 0.013 | 0.09 | 0.692 |
| 12,13-DiHOME * | 25.3 ± 15.4 | 93.7 ± 66.8 | 31.8-292 | −0.47 | 0.033 | 0.10 | 0.680 |
| ²9(10)-EpOME^{#} | 46.3 ± 24.7 | 42.1 ± 27.1 | 4.4-92.7 | 0.02 | 0.931 | −0.49 | 0.026 |
| ²12(13)-EpOME | 50.5 ± 24.7 | 54.1 ± 25.3 | 14.6-100 | −0.16 | 0.483 | −0.28 | 0.214 |
| ²9-HOTrE | 1.1 ± 1.5 | 1.0 ± 1.5 | 0.09-6.3 | −0.27 | 0.232 | 0.17 | 0.460 |
| ²13-HOTrE | 1.2 ± 1.5 | 2.4 ± 3.3 | 0.4-13.0 | −0.35 | 0.118 | −0.10 | 0.678 |
| ³14,15-DiHETrE | 1.6 ± 0.7 | 2.2 ± 0.8 | 1.4-5.4 | −0.28 | 0.211 | −0.17 | 0.461 |

Analytes were quantified by isotopically labeled analogues except for
¹15-HETE-d8,
²9-HODE-d4,
³11,12-DiHETrE-d11
^{a}Correlation of log concentration at day 5 to normalized survival
^{b}Correlation of log concentration at day 5 to normalized recovery
* p < 0.05 and
** p < 0.01 by Pearson correlation to normalized survival
^{#}p < 0.05 by Pearson correlation to normalized recovery

TABLE 4
Post-Transfusion PLT Recovery

| | Correlation | p-value | q-value | Super Pathway | Sub Pathway | Detected, N (%) | |
|---|---|---|---|---|---|---|---|
| Positive Correlations | | | | | | | |
| histidyltryptophan | 0.6785 | 0.0007 | 0.2989 | Peptide | dipeptide | 21 | (100%) |
| dimethylglycine | 0.4885 | 0.0246 | 0.6562 | Amino Acid | Glycine, Serine and | 21 | (100%) |
| | | | | | Threonine | | |
| | | | | | Metabolism | | |
| N1-methyladenosine | 0.4375 | 0.0473 | 0.6784 | Nucleotide | Purine Metabolism, | 17 | (81%) |
| | | | | | Adenine containing | | |
| Negative Correlation | | | | | | | |
| gulonic acid* | −0.4899 | 0.0242 | 0.6562 | Cofactors and | Ascorbate and | 20 | (95%) |
| | | | | Vitamins | Aldarate Metabolism | | |
| 4-vinylphenol sulfate | −0.4565 | 0.0375 | 0.6562 | Xenobiotics | Benzoate | 21 | (100%) |
| | | | | | Metabolism | | |
| 3-methyl catechol | −0.4685 | 0.0322 | 0.6562 | Xenobiotics | Benzoate | 18 | (86%) |
| sulfate (2) | | | | | Metabolism | | |
| 1,2-propanediol | −0.4986 | 0.0214 | 0.6562 | Xenobiotics | Chemical | 21 | (100%) |
| cyclo(gly-pro) | −0.4815 | 0.0271 | 0.6562 | Peptide | Dipeptide | 21 | (100%) |
| cis-Cyclo[L-ala-L-Pro] | −0.6097 | 0.0033 | 0.3345 | Peptide | Dipeptide | 21 | (100%) |
| malonate | −0.481 | 0.0273 | 0.6562 | Lipid | Fatty Acid | 21 | (100%) |
| (propanedioate) | | | | | Synthesis | | |
| undecanedioate | −0.514 | 0.0171 | 0.6506 | Lipid | Fatty Acid, | 21 | (100%) |
| | | | | | Dicarboxylate | | |
| adipate | −0.614 | 0.0031 | 0.3345 | Lipid | Fatty Acid, | 21 | (100%) |
| | | | | | Dicarboxylate | | |
| 2-hydroxypalmitate | −0.485 | 0.0259 | 0.6562 | Lipid | Fatty Acid, | 21 | (100%) |
| | | | | | Monohydroxy | | |
| 1,6-anhydroglucose | −0.5218 | 0.0153 | 0.6279 | Xenobiotics | Food Component/ | 16 | (76%) |
| | | | | | Plant | | |
| gamma-glutamylvaline | −0.4932 | 0.0231 | 0.6562 | Peptide | Gamma-glutamyl | 21 | (100%) |
| | | | | | Amino Acid | | |
| gamma-glutamyltryptophan | −0.5212 | 0.0154 | 0.6279 | Peptide | Gamma-glutamyl | 20 | (95%) |
| | | | | | Amino Acid | | |
| 1-methylhistidine | −0.4556 | 0.0379 | 0.6562 | Amino Acid | Histidine | 21 | (100%) |
| | | | | | Metabolism | | |
| 4-methyl-2-oxopentanote | −0.4439 | 0.0438 | 0.6562 | Amino Acid | Leucine, Isoleucine | 21 | (100%) |
| | | | | | and Valine | | |
| | | | | | Metabolism | | |
| leucine | −0.4448 | 0.0434 | 0.6562 | Amino Acid | Leucine, Isoleucine | 21 | (100%) |
| | | | | | and Valine | | |
| | | | | | Metabolism | | |
| alpha-hydroxyisocaproate | −0.4452 | 0.0431 | 0.6562 | Amino Acid | Leucine, Isoleucine | 21 | (100%) |
| | | | | | and Valine | | |
| | | | | | Metabolism | | |
| isovalerate | −0.461 | 0.0354 | 0.6562 | Amino Acid | Leucine, isoleucine | 21 | (100%) |
| | | | | | and Valine | | |
| | | | | | Metabolism | | |
| alpha-hydroxyisovalerate | −0.5215 | 0.0153 | 0.6279 | Amino Acid | Leucine, Isoleucine | 21 | (100%) |
| | | | | | and Valine | | |
| | | | | | Metabolism | | |
| glutarylcarnitine (C5) | −0.5946 | 0.0045 | 0.3511 | Amino Acid | Lysine Metabolism | 21 | (100%) |
| 2-docosapentaenoylglycero- | −0.4682 | 0.0323 | 0.6562 | Lipid | Lysolipid | 12 | (57%) |
| phosphocholine | | | | | | | |
| (22:5n3)* | | | | | | | |
| palmitoyl-arachidonoyl- | −0.4883 | 0.0247 | 0.6562 | Lipid | Lysolipid | 21 | (100%) |
| glyceropho-sphocholine | | | | | | | |
| (2)* | | | | | | | |
| 1-margaroylglycerophos- | −0.527 | 0.0141 | 0.6279 | Lipid | Lysolipid | 21 | (100%) |
| phocholine (17:0) | | | | | | | |
| 2-stearoylglycerophos- | −0.5529 | 0.0093 | 0.6279 | Lipid | Lysolipid | 21 | (100%) |
| phocholine* | | | | | | | |
| pelargonate (9:0) | −0.4782 | 0.0283 | 0.6562 | Lipid | Medium Chain | 21 | (100%) |
| | | | | | Fatty Acid | | |
| choline phosphate | −0.4433 | 0.0441 | 0.6562 | Lipid | Phospholipid | 21 | (100%) |
| | | | | | Metabolism | | |
| xanthine | −0.5036 | 0.0199 | 0.6562 | Nucleotide | Purine Metabolism, | 21 | (100%) |
| | | | | | (Hypo)Xanthine/ | | |
| | | | | | Inosine containing | | |
| AlCA ribonucleotide | −0.5289 | 0.0137 | 0.6279 | Nucleotide | Purine Metabolism, | 21 | (100%) |
| | | | | | (Hypo)Xanthine/ | | |
| | | | | | Inosine containing | | |
| 3-aminoisobutyrate | −0.4439 | 0.0438 | 0.6562 | Nucleotide | Pyrimidine | 19 | (90%) |
| | | | | | Metabolism, | | |
| | | | | | Thymine containing | | |
| valerate | −0.4894 | 0.0243 | 0.6562 | Lipid | Short Chain | 20 | (95%) |
| | | | | | Fatty Acid | | |
| 4-androsten-3beta,17beta- | −0.4462 | 0.0426 | 0.6562 | Lipid | Steroid | 21 | (100%) |
| diol disulfate (2) | | | | | | | |
| 5-pregnen-3b, | −0.448 | 0.0417 | 0.6562 | Lipid | Steroid | 9 | (43%) |
| 17-diol-20-one 3-sulfate | | | | | | | |
| 4-androsten-3alpha,17alpha- | −0.4643 | 0.034 | 0.6562 | Lipid | Steroid | 20 | (95%) |
| diol monosulfate (2) | | | | | | | |
| 4-androsten-3beta,17beta- | −0.4769 | 0.0288 | 0.6562 | Lipid | Steroid | 21 | (100%) |
| diol monosulfate (1) | | | | | | | |
| 5alpha-androstan- | −0.5106 | 0.018 | 0.6562 | Lipid | Steroid | 19 | (90%) |
| 3beta,17beta-diol | | | | | | | |
| monosulfate (2) | | | | | | | |
| etiocholanolone | −0.6256 | 0.0024 | 0.3345 | Lipid | Steroid | 21 | (100%) |
| glucuronide | | | | | | | |
| indoleacetylglutamine | −0.4572 | 0.0372 | 0.6562 | Amino Acid | Tryptophan | 14 | (67%) |
| | | | | | Metabolism | | |
| indolelactate | −0.4977 | 0.0217 | 0.6562 | Amino Acid | Tryptophan | 21 | (100%) |
| | | | | | Metabolism | | |
| indoleacetate | −0.5216 | 0.0153 | 0.6279 | Amino Acid | Tryptophan | 21 | (100%) |
| | | | | | Metabolism | | |
| caffeine | −0.4832 | 0.0265 | 0.6562 | Xenobiotics | Xanthine | 21 | (100%) |
| | | | | | Metabolism | | |
| 1,3,7-trimethylurate | −0.4951 | 0.0225 | 0.6562 | Xenobiotics | Xanthine | 15 | (71%) |
| | | | | | Metabolism | | |
| paraxanthine | −0.5461 | 0.0104 | 0.6279 | Xenobiotics | Xanthine | 19 | (90%) |
| | | | | | Metabolism | | |
| theophylline | −0.5498 | 0.0098 | 0.6279 | Xenobiotics | Xanthine | 18 | (86%) |
| | | | | | Metabolism | | |
| 5-acetylamino-6-amino- | −0.5965 | 0.0043 | 0.3511 | Xenobiotics | Xanthine | 17 | (81%) |
| 3-methyluracil | | | | | Metabolism | | |
| 1,3-dimethylurate | −0.6097 | 0.0033 | 0.3345 | Xenobiotics | Xanthine | 16 | (76%) |
| | | | | | Metabolism | | |
| 1-methylxanthine | −0.6195 | 0.0027 | 0.3345 | Xenobiotics | Xanthine | 21 | (100%) |
| | | | | | Metabolism | | |
| 1-methylurate | −0.6715 | 0.0009 | 0.2989 | Xenobiotics | Xanthine | 18 | (86%) |
| | | | | | Metabolism | | |
| 1,7-dimethylurate | −0.6777 | 0.0007 | 0.2989 | Xenobiotics | Xanthine | 18 | (86%) |
| | | | | | Metabolism | | |

*Indicates compounds that have not been officially confirmed based on a standard but we are confident in its identity.
Analytes with significant correlations to PLT recoveries. Analytes with p values of less than 0.05 are presented, grouped according to pathways and subpathways. As per main text, q values are shown for each analyte. Permutation analysis for multiple components of the same pathway are presented in the main text. The number (N) and percent (%) of samples with levels detected are indicated for each metabolite. Correlations among metabolites with a low percentage of detected samples are less reliable and should be interpreted with caution.

TABLE 5
Post-Transfusion PLT Survival

| | Correlation | p-value | q-value | Super Pathway | Sub Pathway | Detected, N (%) | |
|---|---|---|---|---|---|---|---|
| Positive Correlations | | | | | | | |
| gamma-glutamyl- | 0.5914 | 0.0047 | 0.2566 | Peptide | Gamma-glutamyl | 18 | (86%) |
| 2-aminobutyrate | | | | | Amino Acid | | |
| bilirubin (Z,Z) | 0.5364 | 0.0122 | 0.4001 | Cofactors and | Hemoglobin and | 21 | (100%) |
| | | | | Vitamins | Porphyrin | | |
| | | | | | Metabolism | | |
| cortisol | 0.5245 | 0.0147 | 0.4269 | Lipid | Steroid | 21 | (100%) |
| tyrosylglutamine | 0.4865 | 0.0253 | 0.5148 | Peptide | Dipeptide | 21 | (100%) |
| eicosanodioate | 0.486 | 0.0255 | 0.5148 | Lipid | Fatty Acid, | 21 | (100%) |
| | | | | | Dicarboxylate | | |
| 2-aminobutyrate | 0.4648 | 0.0337 | 0.5721 | Amino Acid | Methionine, | 21 | (100%) |
| | | | | | Cysteine, SAM | | |
| | | | | | and Taurine | | |
| | | | | | Metabolism | | |
| asparagylvaline | 0.4623 | 0.0348 | 0.582 | Peptide | Dipeptide | 21 | (100%) |
| 21-hydroxypregnenolone | 0.4593 | 0.0362 | 0.5878 | Lipid | Steroid | 21 | (100%) |
| disulfate | | | | | | | |
| 5-pregnen-3b, | 0.4477 | 0.0418 | 0.6049 | Lipid | Steroid | 9 | (43%) |
| 17-diol-20-one | | | | | | | |
| 3-sulfate | | | | | | | |
| 5alpha-pregnan-3beta, | 0.4426 | 0.0445 | 0.6091 | Lipid | Steroid | 20 | (95%) |
| 20beta-diol | | | | | | | |
| monosulfate (1) | | | | | | | |
| 3-methyladipate | 0.4395 | 0.0462 | 0.6105 | Lipid | Fatty Acid, | 12 | (57%) |
| | | | | | Dicarboxylate | | |
| N-acetylisoleucine | 0.4343 | 0.0492 | 0.6339 | Amino Acid | Leucine, | 21 | (100%) |
| | | | | | Isoleucine and | | |
| | | | | | Valine Metabolism | | |
| Negative | | | | | | | |
| Correlations | | | | | | | |
| palmitoyl ethanolamide | −0.4657 | 0.0334 | 0.5721 | Lipid | Endocannabinoid | 21 | (100%) |
| N-palmitoyltaurine | −0.5104 | 0.0181 | 0.4892 | Lipid | Endocannabinoid | 21 | (100%) |
| I inoleoylcarnitine* | −0.4475 | 0.0419 | 0.6049 | Lipid | Fatty Acid | 21 | (100%) |
| | | | | | Metabolism(Acyl | | |
| | | | | | Carnitine) | | |
| decanoylcarnitine | −0.4712 | 0.0311 | 0.5690 | Lipid | Fatty Acid | 21 | (100%) |
| | | | | | Metabolism(Acyl | | |
| | | | | | Carnitine) | | |
| octanoylcarnitine | −0.5080 | 0.0187 | 0.4946 | Lipid | Fatty Acid | 21 | (100%) |
| | | | | | Metabolism(Acyl | | |
| | | | | | Carnitine) | | |
| hexanoylcarnitine | −0.5872 | 0.0051 | 0.2566 | Lipid | Fatty Acid | 21 | (100%) |
| | | | | | Metabolism(Acyl | | |
| | | | | | Carnitine) | | |
| laurylcarnitine | −0.6310 | 0.0022 | 0.2231 | Lipid | Fatty Acid | 21 | (100%) |
| | | | | | Metabolism(Acyl | | |
| | | | | | Carnitine) | | |
| myristoylcarnitine | −0.6908 | 0.0005 | 0.1878 | Lipid | Fatty Acid | 21 | (100%) |
| | | | | | Metabolism(Acyl | | |
| | | | | | Carnitine) | | |
| palmitoylcarnitine | −0.7040 | 0.0004 | 0.1878 | Lipid | Fatty Acid | 21 | (100%) |
| | | | | | Metabolism(Acyl | | |
| | | | | | Carnitine) | | |
| 13-methyl myristic acid | −0.4555 | 0.0380 | 0.5990 | Lipid | Fatty Acid, | 21 | (100%) |
| | | | | | Branched | | |
| 17-methyl stearate | −0.4973 | 0.0218 | 0.5053 | Lipid | Fatty Acid, | 21 | (100%) |
| | | | | | Branched | | |
| 13-HODE + 9-HODE | −0.4891 | 0.0244 | 0.5148 | Lipid | Fatty Acid, | 21 | (100%) |
| | | | | | Monohydroxy | | |
| 3-hydroxymyristate | −0.4987 | 0.0214 | 0.5053 | Lipid | Fatty Acid, | 21 | (100%) |
| | | | | | Monohydroxy | | |
| glycerol | −0.4870 | 0.0252 | 0.5148 | Lipid | Glycerolipid | 21 | (100%) |
| | | | | | Metabolism | | |
| glycerol 3-phosphate | −0.5497 | 0.0098 | 0.3584 | Lipid | Glycerolipid | 21 | (100%) |
| (G3P) | | | | | Metabolism | | |
| stearate (18:0) | −0.4594 | 0.0362 | 0.5878 | Lipid | Long Chain | 21 | (100%) |
| | | | | | Fatty Acid | | |
| cis-vaccenate (18:1n7) | −0.4672 | 0.0327 | 0.5717 | Lipid | Long Chain | 21 | (100%) |
| | | | | | Fatty Acid | | |
| margarate (17:0) | −0.4797 | 0.0278 | 0.5260 | Lipid | Long Chain | 21 | (100%) |
| | | | | | Fatty Acid | | |
| pentadecanoate (15:0) | −0.5182 | 0.0161 | 0.4572 | Lipid | Long Chain | 21 | (100%) |
| | | | | | Fatty Acid | | |
| arachidate (20:0) | −0.5308 | 0.0133 | 0.4086 | Lipid | Long Chain | 21 | (100%) |
| | | | | | Fatty Acid | | |
| myristoleate (14:1n5) | −0.5315 | 0.0131 | 0.4086 | Lipid | Long Chain | 9 | (43%) |
| | | | | | Fatty Acid | | |
| 10-nonadecenoate | −0.5357 | 0.0123 | 0.4001 | Lipid | Long Chain | 21 | (100%) |
| (19:1n9) | | | | | Fatty Acid | | |
| nonadecanoate (19:0) | −0.5831 | 0.0055 | 0.2616 | Lipid | Long Chain | 21 | (100%) |
| | | | | | Fatty Acid | | |
| myristate (14:0) | −0.5865 | 0.0052 | 0.2566 | Lipid | Long Chain | 21 | (100%) |
| | | | | | Fatty Acid | | |
| 10-heptadecenoate | −0.5965 | 0.0043 | 0.2451 | Lipid | Long Chain | 21 | (100%) |
| (17:1n7) | | | | | Fatty Acid | | |
| palmitate (16:0) | −0.5993 | 0.0041 | 0.2445 | Lipid | Long Chain | 21 | (100%) |
| | | | | | Fatty Acid | | |
| oleate (18:1n9) | −0.6098 | 0.0033 | 0.2327 | Lipid | Long Chain | 21 | (100%) |
| | | | | | Fatty Acid | | |
| palmitoleate (16:1n7) | −0.6342 | 0.0020 | 0.2231 | Lipid | Long Chain | 21 | (100%) |
| | | | | | Fatty Acid | | |
| erucate (22:1n9) | −0.6483 | 0.0015 | 0.2231 | Lipid | Long Chain | 21 | (100%) |
| | | | | | Fatty Acid | | |
| eicosenoate (20:1n9 or | −0.6570 | 0.0012 | 0.2231 | Lipid | Long Chain | 21 | (100%) |
| 11) | | | | | Fatty Acid | | |
| 1-palmitoylglycero- | −0.4558 | 0.0378 | 0.5990 | Lipid | Lysolipid | 21 | (100%) |
| phosphocholine (16:0) | | | | | | | |
| 1-docosapentaenoylglycero- | −0.5029 | 0.0201 | 0.5050 | Lipid | Lysolipid | 21 | (100%) |
| phosphocholine (22:5n6)* | | | | | | | |
| 1-stearoylglyceropho- | −0.5137 | 0.0172 | 0.4772 | Lipid | Lysolipid | 21 | (100%) |
| phoinositol | | | | | | | |
| 1-nonadecanoylglycero- | −0.5635 | 0.0078 | 0.3167 | Lipid | Lysolipid | 21 | (100%) |
| phosphocholine(19:0) | | | | | | | |
| 1-docosapentaenoylglycero- | −0.5656 | 0.0075 | 0.3167 | Lipid | Lysolipid | 21 | (100%) |
| phosphocholine (22:5n3)* | | | | | | | |
| 1-eicosatrienoylglycero- | −0.6331 | 0.0021 | 0.2231 | Lipid | Lysolipid | 21 | (100%) |
| phosphocholine (20:3)* | | | | | | | |
| caprate (10:0) | −0.4519 | 0.0397 | 0.6049 | Lipid | Medium Chain | 21 | (100%) |
| | | | | | Fatty Acid | | |
| 5-dodecenoate | −0.5713 | 0.0068 | 0.2983 | Lipid | Medium Chain | 21 | (100%) |
| (12:1n7) | | | | | Fatty Acid | | |
| laurate (12:0) | −0.6432 | 0.0017 | 0.2231 | Lipid | Medium Chain | 21 | (100%) |
| | | | | | Fatty Acid | | |
| 3-hydroxy-3-methylglutarate | −0.5003 | 0.0209 | 0.5050 | Lipid | Mevalonate | 21 | (100%) |
| | | | | | Metabolism | | |
| 1-oleoylglycerol | −0.5734 | 0.0066 | 0.2983 | Lipid | Monoacylglycerol | 21 | (100%) |
| (1-monoolein) | | | | | | | |
| thyroxine | −0.6076 | 0.0035 | 0.2327 | Amino Acid | Phenylalanine | 21 | (100%) |
| | | | | | and Tyrosine | | |
| | | | | | Metabolism | | |
| glycerophosphoinositol* | −0.4851 | 0.0258 | 0.5148 | Lipid | Phospholipid | 21 | (100%) |
| | | | | | Metabolism | | |
| docosahexaenoate | −0.4402 | 0.0458 | 0.6105 | Lipid | Polyunsaturated | 21 | (100%) |
| (DHA; 22:6n3) | | | | | Fatty Acid (n3 | | |
| | | | | | and n6) | | |
| arachidonate (20:4n6) | −0.4442 | 0.0437 | 0.6049 | Lipid | Polyunsaturated | 21 | (100%) |
| | | | | | Fatty Acid (n3 | | |
| | | | | | and n6) | | |
| eicosapentaenoate | −0.4462 | 0.0426 | 0.6049 | Lipid | Polyunsaturated | 21 | (100%) |
| (EPA; 20:5n3) | | | | | Fatty Acid (n3 | | |
| | | | | | and n6) | | |
| linolenate [alpha or | −0.4880 | 0.0248 | 0.5148 | Lipid | Polyunsaturated | 21 | (100%) |
| gamma; (18:3n3 or 6)] | | | | | Fatty Acid (n3 | | |
| | | | | | and n6) | | |
| dihomo-linolenate | −0.5267 | 0.0142 | 0.4236 | Lipid | Polyunsaturated | 21 | (100%) |
| (20:3n3 or n6) | | | | | Fatty Acid (n3 | | |
| | | | | | and n6) | | |
| docosapentaenoate | −0.5462 | 0.0104 | 0.3584 | Lipid | Polyunsaturated | 21 | (100%) |
| (n6 DPA; 22:5n6) | | | | | Fatty Acid (n3 | | |
| | | | | | and n6) | | |
| docosadienoate | −0.5599 | 0.0083 | 0.3250 | Lipid | Polyunsaturated | 21 | (100%) |
| (22:2n6) | | | | | Fatty Acid (n3 | | |
| | | | | | and n6) | | |
| docosatrienoate | −0.6118 | 0.0032 | 0.2327 | Lipid | Polyunsaturated | 21 | (100%) |
| (22:3n3) | | | | | Fatty Acid (n3 | | |
| | | | | | and n6) | | |
| linoleate (18:2n6) | −0.6262 | 0.0024 | 0.2263 | Lipid | Polyunsaturated | 21 | (100%) |
| | | | | | Fatty Acid (n3 | | |
| | | | | | and n6) | | |
| dihomo-linoleate | −0.6820 | 0.0007 | 0.1878 | Lipid | Polyunsaturated | 21 | (100%) |
| (20:2n6) | | | | | Fatty Acid (n3 | | |
| | | | | | and n6) | | |
| docosapentaenoate | −0.7011 | 0.0004 | 0.1878 | Lipid | Polyunsaturated | 21 | (100%) |
| (n3 DPA; 22:5n3) | | | | | Fatty Acid (n3 | | |
| | | | | | and n6) | | |
| guanosine | −0.5007 | 0.0208 | 0.5050 | Nucleotide | Purine | 21 | (100%) |
| | | | | | Metabolism, | | |
| | | | | | Guanine | | |
| | | | | | containing | | |
| sphinganine | −0.5059 | 0.0193 | 0.4983 | Lipid | Sphingolipid | 21 | (100%) |
| | | | | | Metabolism | | |
| gamma-tocopherol | −0.5535 | 0.0092 | 0.3498 | Cofactors and | Tocopherol | 21 | (100%) |
| | | | | Vitamins | Metabolism | | |

Analytes with significant correlations to PLT survivals. Analytes with p values of less than 0.05 are presented, grouped according to pathways and subpathways. As per main text, q values are shown for each analyte. Permutation analysis for multiple components of the same pathway are presented in the main text. The number (N) and percent (%) of samples with levels detected are indicated for each metabolite. Correlations among metabolites with a low percentage of detected samples are less reliable and should be interpreted with caution.

While specific aspects of the invention have been described and illustrated, such aspects should be considered illustrative of the invention only and not as limiting the invention as construed in accordance with the accompanying claims.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed:

1. A method of determining post-transfusion survival of platelets (PLT) prior to transfusion, the method comprising the steps of:
a) measuring the levels of one or more biochemical mediators in a PLT sample selected from the group consisting of the compounds in Tables 2-5;
b) comparing the level of the one or more biochemical mediators in the PLT sample with the level of the one or more biochemical mediators present in a control sample;
c) determining a higher or lower level of the one or more biochemical mediators in the PLT sample that indicates the post-transfusion survival of platelets in the PLT sample.

2. The method of claim 1, further comprising selecting the PLT sample for use in a transfusion when a higher post-transfusion survival is indicated.

3. The method of claim 1, further comprising excluding the PLT sample for use in a transfusion when a lower post-transfusion survival is indicated.

4. A method of determining the suitability of a platelet (PLT) unit for transfusion, the method comprising the steps of: a) measuring the levels of one or more biochemical mediators in a PLT sample selected from the group consisting of the compounds in Tables 2-5;
b) comparing the level of the one or more biochemical mediators in the PLT sample with the level of the one or more biochemical mediators present in a control sample;
c) determining a higher or lower level of the one or more biochemical mediators in the PLT sample that indicates the suitability of the platelet (PLT) unit for transfusion.

5. The method of claim 4, further comprising selecting the PLT sample for use in a transfusion when a higher suitability of the platelet (PLT) unit for transfusion is indicated.

6. The method of claim 4, further comprising excluding the PLT sample for use in a transfusion when a lower suitability of the platelet (PLT) unit for transfusion is indicated.

7. The method of claim 4, wherein the levels of the one or more biochemical mediators in the PLT sample is indicative of the level of leukotrienes or prostaglandins in the PLT sample, thereby indicating the suitability of the sample for transfusion.

8. The method of claim 1 or 4, wherein the measurement is performed at the time of collection of the PLT sample.

9. The method of claim 1 or 4, wherein the measurement is performed during the time of storage of the PLT sample.

10. The method of claim 1 or 4, wherein the measurement is performed by mass spectrometry.

11. The method of claim 10, wherein the mass spectrometry is gas-chromatography/mass spectrometry (GC/MS) or liquid chromatography-tandem mass spectrometry (LC/MS/MS).

12. The method of claim 1 or 4, wherein the measurement is performed by enzymatic assay.

13. The method of claim 1 or 4, wherein the measurement is performed by ELISA.

14. The method of claim 1 or 4, wherein the level of the one or more biochemical mediator is 2-200 fold higher than in the control sample.

15. A method for determining PLT storage quality, the method comprising the steps of:
obtaining a dataset associated with a sample of stored platelets, wherein the dataset comprises at least one biochemical mediator, selected from the group consisting of the compounds in Tables 2-5;
analyzing the dataset to determine data for the at least one biochemical mediator,
wherein the data is positively correlated or negatively correlated with PLT storage quality of the sample of stored platelets.

16. The method of claim 15, wherein the sample of stored platelets is selected for use in a transfusion when the data is positively correlated correlated with PLT storage quality of the sample of stored platelets.

17. The method of claim 15, wherein the sample of stored platelets is excluded from use in a transfusion when the data is is negatively correlated correlated with PLT storage quality of the sample of stored platelets.

18. A method for determining PLT storage quality, the method comprising the steps of:
obtaining a sample of stored platelets, wherein the sample comprises at least one biochemical mediator, selected from the group consisting of the compounds in Tables 2-5;
contacting the sample with a reagent;
generating a complex between the reagent and the at least one biochemical mediator;
detecting the complex to obtain a dataset associated with the sample, wherein the dataset comprises expression or activity level data for the at least one biochemical mediator; and
analyzing the expression or activity level data for the at least one biochemical mediator,
wherein the expression or activity level of the at least one biochemical mediator is positively correlated or negatively correlated with PLT storage quality.

19. The method of claim 18, wherein the sample of stored platelets is selected for use in a transfusion when the data is positively correlated with PLT storage quality of the sample of stored platelets.

20. The method of claim 18, wherein the sample of stored platelets is excluded from use in a transfusion when the data is negatively correlated correlated with PLT storage quality of the sample of stored platelets.

21. A computer-implemented method for determining PLT storage quality, the method comprising the steps of:
storing, in a storage memory, a dataset associated with a stored platelet sample, wherein the dataset comprises data for at least one biochemical mediator, selected from the group consisting of the compounds in Tables 2-5; and
analyzing, by a computer processor, the dataset to determine the expression or activity levels of the at least one biochemical mediator, wherein the expression or activity levels are positively correlated or negatively correlated with PLT storage quality.

22. The method of claim 21, wherein the sample of stored platelets is selected for use in a transfusion when the data is positively correlated with PLT storage quality of the sample of stored platelets.

23. The method of claim 21, wherein the sample of stored platelets is excluded from use in a transfusion when the data is negatively correlated correlated with PLT storage quality of the sample of stored platelets.

24. A system for determining PLT storage quality, the system comprising:
a storage memory for storing a dataset associated with a stored platelet sample, wherein the dataset comprises data for at least one biochemical mediator, wherein the dataset comprises data for at least one biochemical mediator, selected from the group consisting the compounds in Tables 2-5; and
a processor communicatively coupled to the storage memory for analyzing the dataset to determine the activity or expression levels of the at least one biochemical mediator, wherein the activity or expression levels are positively correlated or negatively correlated with PLT storage quality.

25. The method of claim 24, wherein the sample of stored platelets is selected for use in a transfusion when the data is positively correlated with PLT storage quality of the sample of stored platelets.

26. The method of claim 24, wherein the sample of stored platelets is excluded from use in a transfusion when the data is negatively correlated correlated with PLT storage quality of the sample of stored platelets.

27. A computer-readable storage medium storing computer-executable program code, the program code comprising:
program code for storing a dataset associated with a stored platelet sample, wherein the dataset comprises data for at least one biochemical mediator, wherein the dataset comprises data for at least one biochemical mediator, selected from the group consisting of the compounds in Tables 2-5; and
program code for analyzing the dataset to determine the activity or expression levels of the at least one biochemical mediator, wherein the activity or expression levels of the biochemical mediators are positively correlated or negatively correlated with PLT storage quality.

28. The method of claim 27, wherein the sample of stored platelets is selected for use in a transfusion when the data is positively correlated with PLT storage quality of the sample of stored platelets.

29. The method of claim 27, wherein the sample of stored platelets is excluded from use in a transfusion when the data is negatively correlated correlated with PLT storage quality of the sample of stored platelets.

30. A method for predicting transfusion outcome, the method comprising the steps of:
obtaining a dataset associated with a sample of stored platelets, wherein the dataset comprises at least one biochemical mediator, wherein the dataset comprises data for at least one biochemical mediator, selected from the group consisting of the compounds in Tables 2-5;
analyzing the dataset to determine data for the at least one biochemical mediator, wherein the data is positively correlated or negatively correlated with transfusion outcome if the platelet sample is transfused into a patient.

31. The method of claim 30, wherein the sample of stored platelets is selected for use in a transfusion when the data is positively correlated with transfusion outcome if the sample of stored platelets is transfused into a patient.

32. The method of claim 30, wherein the sample of stored platelets is excluded from use in a transfusion when the data is negatively correlated with transfusion outcome if the sample of stored platelets is transfused into a patient.

33. A method for predicting transfusion outcome, the method comprising the steps of:
obtaining a sample of stored platelets, wherein the sample comprises at least one biochemical mediator, wherein the dataset comprises data for at least one biochemical mediator, selected from the group consisting of the compounds in Tables 2-5;
contacting the sample with a reagent;
generating a complex between the reagent and the at least one biochemical mediator;
detecting the complex to obtain a dataset associated with the sample, wherein the dataset comprises expression or activity level data for the at least one biochemical mediator; and
analyzing the expression or activity level data for the biochemical mediators, wherein the expression or activity level of the at least one biochemical mediator is positively correlated or negatively correlated with transfusion outcome if the platelet sample is transfused into a patient.

34. The method of claim 33, wherein the sample of stored platelets is selected for use in a transfusion when the data is positively correlated with transfusion outcome if the sample of stored platelets is transfused into a patient.

35. The method of claim 33, wherein the sample of stored platelets is excluded from use in a transfusion when the data is negatively correlated with transfusion outcome if the sample of stored platelets is transfused into a patient.

36. A computer-implemented method for predicting transfusion outcome, the method comprising the steps of:
storing, in a storage memory, a dataset associated with a stored platelet sample, wherein the dataset comprises data for at least one biochemical mediator wherein the dataset comprises data for at least one biochemical mediator, selected from the group consisting of the compounds in Tables 2-5; and
analyzing, by a computer processor, the dataset to determine the expression or activity levels of the at least one biochemical mediator, wherein the expression or activity levels are positively correlated or negatively correlated with transfusion outcome if the platelet sample is transfused into a patient.

37. The method of claim 36, wherein the sample of stored platelets is selected for use in a transfusion when the data is positively correlated with transfusion outcome if the sample of stored platelets is transfused into a patient.

38. The method of claim 36, wherein the sample of stored platelets is excluded from use in a transfusion when the data is negatively correlated with transfusion outcome if the sample of stored platelets is transfused into a patient.

39. A system for predicting transfusion outcome, the system comprising:
a storage memory for storing a dataset associated with a stored platelet sample, wherein the dataset comprises data for at least one biochemical mediator, wherein the dataset comprises data for at least one biochemical mediator, selected from the group consisting of the compounds in Tables 2-5; and
a processor communicatively coupled to the storage memory for analyzing the dataset to determine the activity or expression levels of the at least one biochemical mediator, wherein the activity or expression levels are positively correlated or negatively correlated with transfusion outcome if the platelet sample is transfused into a patient.

40. The method of claim 39, wherein the sample of stored platelets is selected for use in a transfusion when the data is positively correlated with transfusion outcome if the sample of stored platelets is transfused into a patient.

41. The method of claim 39, wherein the sample of stored platelets is excluded from use in a transfusion when the data is negatively correlated with transfusion outcome if the sample of stored platelets is transfused into a patient.

42. A computer-readable storage medium storing computer-executable program code, the program code comprising:
program code for storing a dataset associated with a stored platelet sample, wherein the dataset comprises data for at least one biochemical mediator, selected from the group consisting of the compounds in Tables 2-5; and
program code for analyzing the dataset to determine the activity or expression levels of the at least one marker, wherein the activity or expression levels of the biochemical mediators are positively correlated or negatively correlated with transfusion outcome if the platelet sample is transfused into a patient.

43. The method of claim 42, wherein the sample of stored platelets is selected for use in a transfusion when the data is positively correlated with transfusion outcome if the sample of stored platelets is transfused into a patient.

44. The method of claim 42, wherein the sample of stored platelets is excluded from use in a transfusion when the data is negatively correlated with transfusion outcome if the sample of stored platelets is transfused into a patient.

45. The method or storage medium or system of any one of claims 15-44, wherein the dataset is obtained at the time of collection of the PLT sample.

46. The method or storage medium or system of any one of claims 15-44, wherein the dataset is obtained during the time of storage of the PLT sample.

47. The method or storage medium or system of any one of claims 15-44, wherein the dataset is obtained by mass spectrometry.

48. The method of claim 47, wherein the mass spectrometry is gas-chromatography/mass spectrometry (GC/MS) or liquid chromatography-tandem mass spectrometry (LC/MS/MS).

49. The method or storage medium or system of any one of claims 15-44, wherein the dataset is obtained by enzymatic assay.

50. The method or storage medium or system of any one of claims 15-44, wherein the dataset is obtained by ELISA.

51. A kit for use in predicting transfusion outcome or platelet (PLT) storage quality, the kit comprising:
a set of reagents comprising a plurality of reagents for determining from a stored platelet sample data for at least one biochemical mediator, selected from the group consisting of the compounds in Tables 2-5; and
instructions for using the plurality of reagents to determine data from the stored platelet sample.
